(19) United States
(12) Patent Application Publication
(10) Pub. No.: US 2016/0298157 A1
(43) Pub. Date: Oct. 13, 2016

(54) COMPOSITIONS COMPRISING A BETA-GLUCOSIDASE POLYPEPTIDE AND METHODS OF USE

(71) Applicant: DANISCO US INC., Palo Alto, CA (US)

(72) Inventors: Benjamin S. BOWER, Newark, CA (US); Jimmy CHAN, Mountain View, CA (US); Meredith K. FUJDALA, San Jose, CA (US)

(21) Appl. No.: 15/038,587

(22) PCT Filed: Nov. 20, 2014
(86) PCT No.: PCT/US14/66605

§ 371 Date: May 23, 2016

Related U.S. Application Data
(60) Provisional application No. 61/911,722, filed on Dec. 4, 2013.

(51) Int. Cl. C12P 19/14; C12P 19/02; C12N 9/42

(52) U.S. Cl. CPC C12P 19/14; C12N 9/2445; C12Y 302/01021; C12P 19/02

(57) ABSTRACT

The present compositions and methods relate to a beta-glucosidase from Melanocarpus albomyces, polynucleotides encoding the beta-glucosidase, and methods of making and/or using the same. Formulations containing the beta-glucosidase are suitable for use in numerous applications, including hydrolyzing lignocellulosic biomass substrates.

### CROSS REFERENCE TO RELATED APPLICATIONS

This application claims benefit of priority from U.S. Provisional Application No. 61/911,722 filed on 4 Dec. 2013, and the contents of which are incorporated herein by reference in entirety.

### TECHNICAL FIELD

The present compositions and methods relate to a beta-glucosidase polypeptide obtainable from the thermophilic fungus Melanocarpus albomyces, polynucleotides encoding the beta-glucosidase polypeptide, and methods of making and using thereof. Formulations and compositions comprising the beta-glucosidase polypeptide are useful for degrading or hydrolyzing lignocellulosic biomass.

### BACKGROUND

Cellulose and hemicellulose are the most abundant plant materials produced by photosynthesis. They can be degraded and used as an energy source by numerous microorganisms (e.g., bacteria, yeast and fungi) that produce extracellular enzymes capable of hydrolysis of the polymeric substrates to monomeric sugars (Aro et al., J. Biol. Chem., 276: 24309-24314, 2001). As the limits of non-renewable resources approach, the potential of cellulose to become a major renewable energy resource is enormous (Krishna et al., Bioresource Tech., 77: 193-196, 2001). The effective utilization of cellulose through biological processes is one approach to overcoming the shortage of foods, feeds, and fuels (Ohmiya et al., Biotechnol. Gen. Engineer Rev., 14: 365-414, 1997).

Cellulases are enzymes that hydrolyze cellulose (comprising beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”) (Knowles et al., TIBTECH 5: 255-261, 1987; and Schulein, Methods Enzymol., 160: 234-243, 1988). Endoglucanases act mainly on the amorphous parts of the cellulose fiber, whereas cellobiohydrolases are also able to degrade crystalline cellulose (Nevalainen and Penttila, Mycota, 303-319, 1995). Thus, the presence of a cellobiohydrolase in a cellulase system is required for efficient solubilization of crystalline cellulose (Suurnakki et al., Cellulose, 7: 189-209, 2000). Beta-glucosidase acts to liberate β-D-glucose units from cellobiose, cello-oligosaccharides, and other glucosides (Freer, J. Biol. Chem., 268: 9337-9342, 1993).

Cellulases are known to be produced by a large number of bacteria, yeast and fungi. Certain fungi produce a complete cellulase system capable of degrading crystalline forms of cellulose. These fungi can be fermented to produce suites of cellulases or cellulase mixtures. The same fungi and other fungi can also be engineered to produce or overproduce certain cellulases, resulting in mixtures of cellulases that comprise different types or proportions of cellulases. The fungi can also be engineered such that they produce in large quantities via fermentation the various cellulases. Filamentous fungi play a special role since many yeast, such as Saccharomyces cerevisiae, lack the ability to hydrolyze cellulose in their native state (see, e.g., Wood et al., Methods in Enzymology, 160: 87-116, 1988).

The fungal cellulase classifications of CBH, EG and BG can be further expanded to include multiple components within each classification. For example, multiple CBHs, EGs and BGs have been isolated from a variety of fungal sources including Trichoderma reesei (T. reesei, also referred to as Hypocrea jecorina), which contains known genes for two CBHs, i.e., CBH I (“CBH1”) and CBH II (“CBH2”), at least six EGs, e.g., EG I, EG II, EG III, EGV, EGVI, and EGVIII, at least five BGs, e.g., BG1, BG2, BG3, BG4, BG5 and BG7 (Foreman et al. (2003), J. Biol. Chem. 278(34):31988-31997).

In addition to the cellulases above, enzymes having “auxiliary activity” in degrading cellulose have been identified. Examples of enzymes having auxiliary activity include lytic polysaccharide mono-oxygenases (LPMO), for example GH61A and GH61B. These enzymes are now classified as Auxiliary Activity Family 9 (AA9) enzymes (see Hemsworth et al., Current Opinion in Structural Biology (2013) vol. 23, issue 5, pp. 660-668). Additionally, an Auxiliary Activity Family 10 (AA10) has been formed that includes LPMOs that were formerly referred to as CBM33 enzymes, with some members acting on cellulose and some on chitin.

In order to efficiently convert crystalline cellulose to glucose the complete cellulase system comprising components from each of the CBH, EG and BG classifications is required, with isolated components less effective in hydrolyzing crystalline cellulose (Filho et al., Can. J. Microbiol., 42:1-5, 1996). Endo-1,4-beta-glucanases (EG) and exo-cellobiohydrolases (CBH) catalyze the hydrolysis of cellulose to cellooligosaccharides (cellobiose as a main product), while beta-glucosidases (BGL) convert the oligosaccharides to glucose. A synergistic relationship has been observed among cellulase components from different classifications. In particular, the EG-type cellulases and CBH-type cellulases synergistically interact to efficiently degrade cellulose. The beta-glucosidases serve the important role of liberating glucose from the cello-oligosaccharides such as cellobiose, which is inhibitory to the activities of endoglucanases and cellobiohydrolases, thus rendering them ineffective in further hydrolyzing the crystalline cellulose.

In view of the important role played by beta-glucosidases in the degradation or conversion of cellulosic materials, the discovery, characterization, preparation, and application of beta-glucosidase homologs with improved efficacy or capability (or other functional property) to hydrolyze cellulosic feedstock is desirable and advantageous, including those derived from thermophilic fungi.

### SUMMARY

Enzymatic hydrolysis of cellulose remains one of the main limiting steps of the biological production from lignocellulosic biomass feedstock of a material, which may be cellulosic sugars and/or downstream products. Beta-glucosidases play the important role of catalyzing the last step of that process, releasing glucose from the inhibitory cellobiose, and therefore its activity and efficacy directly contributes to the overall efficacy of enzymatic lignocellulosic biomass conversion, and consequently to the cost in use of the enzyme solution. Accordingly there is great interest in finding, making and using new and more effective beta-glucosidases.

While a number of beta-glucosidases are known, including the beta-glucosidases Bgl1, Bgl3, Bgl5, Bgl7, etc., from Trichoderma reesei or Hypocrea jecorina (Korotkova O. G. et al., Biochemistry 74:569-577 (2009); Chauve, M. et al., Biotechnol. Biofuels 3:3-3 (2010)), the beta-glucosidases from Humicola grisea var. thermoidea (Nascimento, C. V. et al., J. Microbiol. 48, 53-62 (2010)); from Sporotrichum pulverulentum, Deshpande V. et al., Methods Enzymol., 160:415-424 (1988)); of Aspergillus oryzae (Fukuda T. et al, Appl. Microbiol. Biotechnol. 76:1027-1033 (2007), from Talaromyces thermophilus CBS 236.58 (Nakkharat P. et al., J. Biotechnol., 123:304-313 (2006)), from Talaromyces emersonii (Murray P., et al, Protein Expr. Purif. 38:248-257 (2004)), so far the Trichoderma reesei beta-glucosidase Bgl1 and the Aspergillus niger beta-glucosidase SP188 are deemed benchmark beta-glucosidases against which the activities and performance of other beta-glucosidases are evaluated. It has been reported that Trichoderma reesei Bgl1 has higher specific activity than Aspergillus niger beta-glucosidase SP188, but the former can be poorly secreted, while the latter is more sensitive to glucose inhibition (Chauve, M. et al., Biotechnol. Biofuels, 3(1):3 (2010)).

Aspects of the present disclosure relates to composition and methods pertaining to beta-glucosidase polypeptides of glycosyl hydrolase family 3 (GH3) derived from the thermophilic filamentous fungus Melanocarpus albomyces (referred to herein as “Mal3A” or “Mal3A polypeptides”), nucleic acids encoding the same, compositions comprising the same, and methods of using such Mal3A polypeptides (and compositions containing them) in hydrolyzing or converting lignocellulosic biomass into soluble, fermentable sugars. Such fermentable sugars can then be converted into cellulosic ethanol, fuels, and other biochemicals and useful products. In certain embodiments, the Mal3A beta-glucosidase polypeptides have higher beta-glucosidase activity and/or exhibit an increased capacity to hydrolyze a given lignocellulosic biomass substrate as compared to the benchmark Trichoderma reesei Bgl1, which is a known, high fidelity beta-glucosidase (Chauve, M. et al., Biotechnol. Biofuels, 3(1):3 (2010)).

In some embodiments, a Mal3A polypeptide is applied together with, or in the presence of, one or more other cellulases in an enzyme composition to hydrolyze or breakdown a suitable biomass substrate. The one or more other cellulases may be, for example, other beta-glucosidases, cellobiohydrolases, and/or endoglucanases. For example, the enzyme composition can contain a Mal3A polypeptide, a cellobiohydrolase, and an endoglucanase (and optionally other components). In some embodiments, the Mal3A polypeptide is applied together with, or in the presence of, one or more hemicellulases in an enzyme composition. The one or more hemicellulases may be, for example, xylanases, beta-xylosidases, and/or α-L-arabinofuranosidases. In further embodiments, the Mal3A polypeptide is applied together with, or in the presence of, one or more cellulases, one or more hemicellulases, and/or one or more auxiliary enzymes in an enzyme composition. For example, the enzyme composition can contain a Mal3A polypeptide and at least one additional enzyme selected from: one or more other beta-glucosidases, one or more cellobiohydrolases, one or more endoglucanases; one or more xylanases, one or more beta-xylosidases, one or more α-L-arabinofuranosidases, and/or one or more lytic polysaccharide mono-oxygenases (LPMO) (e.g., AA9 or AA10 enzymes as described in Hemsworth et al., Current Opinion in Structural Biology (2013) vol. 23, issue 5, pp. 660-668).

In certain embodiments, a Mal3A polypeptide, or a composition comprising the Mal3A polypeptide, is applied to a lignocellulosic biomass substrate or a partially hydrolyzed lignocellulosic biomass substrate in the presence of an ethanologen microbe, which is capable of metabolizing the soluble fermentable sugars produced by the enzymatic hydrolysis of the lignocellulosic biomass substrate, and converting such sugars into ethanol, biochemicals or other useful materials. Such a process may be a strictly sequential process whereby the hydrolysis step occurs before the fermentation step. Such a process may, alternatively, be a hybrid process, whereby the hydrolysis step starts first but for a period overlaps the fermentation step, which starts later. Such a process may, in a further alternative, be a simultaneous hydrolysis and fermentation process, whereby the enzymatic hydrolysis of the biomass substrate occurs while the sugars produced from the enzymatic hydrolysis are fermented by the ethanologen.

The Mal3A polypeptide, for example, may be a part of an enzyme composition, contributing to the enzymatic hydrolysis process and to the liberation of D-glucose from oligosaccharides such as cellobiose. In certain embodiments, the Mal3A polypeptide may be genetically engineered to be expressed in an ethanologen, such that the ethanologen microbe expresses and/or secrets the Mal3A thereby contributing beta-glucosidase activity. Moreover, the Mal3A polypeptide may be a part of the hydrolysis enzyme composition while at the same time may also be expressed and/or secreted by the ethanologen. In such an embodiment, the soluble fermentable sugars produced by the hydrolysis of the lignocellulosic biomass substrate using the hydrolysis enzyme composition is metabolized and/or converted into ethanol by an ethanologen microbe that also expresses and/or secrets the Mal3A polypeptide. The hydrolysis enzyme composition can comprise the Mal3A polypeptide in addition to one or more other cellulases and/or one or more hemicellulases. The ethanologen can be engineered such that it expresses the Mal3A polypeptide, one or more other cellulases, one or more other hemicellulases, or a combination of these enzymes. One or more of the beta-glucosidases may be in the hydrolysis enzyme composition and expressed and/or secreted by the ethanologen. For example, the hydrolysis of the lignocellulosic biomass substrate may be achieved using an enzyme composition comprising a Mal3A polypeptide, and the sugars produced from the hydrolysis can then be fermented with a microorganism engineered to express and/or secret a Mal3A polypeptide. Alternatively, an enzyme composition comprising a first beta-glucosidase participates in the hydrolysis step and a second beta-glucosidase, which is different from the first beta-glucosidase, is expressed and/or secreted by the ethanologen during fermentation. For example, the hydrolysis of the lignocellulosic biomass substrate may be achieved using a hydrolysis enzyme composition comprising T. reesei Bgl1, and the fermentable sugars produced from hydrolysis are fermented by an ethanologen microorganism expressing and/or secreting a Mal3A polypeptide, or vice versa.

As demonstrated herein, Mal3A polypeptides and compositions comprising Mal3A polypeptides have improved efficacy at conditions under which saccharification and degradation of lignocellulosic biomass take place. The improved efficacy of an enzyme composition comprising a Mal3A polypeptide is shown when its performance of hydrolyzing a given biomass substrate is compared to that of an otherwise comparable enzyme composition comprising Bgl1 of T. reesei.

In certain embodiments, the improved or increased beta-glucosidase activity is reflected in an improved or increased cellobiase activity of the Mal3A polypeptides, which is measured using cellobiose as substrate, for example, at a temperature of about 30° C. to about 65° C. (e.g., about 35° C. to about 60° C., about 40° C. to about 55° C., about 45° C. to about 55° C., about 48° C. to about 52° C., about 40° C., about 45° C., about 50° C., about 55° C., etc). In some embodiments, the improved beta-glucosidase activity of a Mal3A polypeptide as compared to that of T. reesei Bgl1 is observed when the beta-glucosidase polypeptides are used to hydrolyze a phosphoric acid swollen cellulose (PASC), for example, pretreated Avicel using an adapted protocol of Walseth (TAPPI 1971, 35:228 and Wood, Biochem. J. 1971, 121:353-362). In some embodiments, the improved beta-glucosidase activity of a Mal3A polypeptide as compared to that of T. reesei Bgl1 is observed when the beta-glucosidase polypeptides are used to hydrolyze a dilute ammonia pretreated corn stover (daCS), for example, one described in International Published Patent Applications: WO2006110891, WO2006110899, WO2006110900, WO2006110901, WO2006110902, and/or in U.S. Pat. Nos. 7,998,713 and 7,932,063.

In some aspects, a Mal3A polypeptide and/or as it is applied in an enzyme composition or in a method to hydrolyze a lignocellulosic biomass substrate is (a) derived from, obtainable from, or produced by Melanocarpus albomyces; (b) a recombinant polypeptide comprising an amino acid sequence that is at least 75% (e.g., at least 75%, 80%, 85%, 90%, 91%, 92%, 93%, 94%, 95%, 96%, 97%, 98%, 99%, or even 100%) identical to the full length amino acid sequence of Mal3A shown in SEQ ID NO:2; (c) a recombinant polypeptide comprising an amino acid sequence that is at least 75% (e.g., at least 75%, 80%, 85%, 90%, 91%, 92%, 93%, 94%, 95%, 96%, 97%, 98%, 99%, or 100%) identical to the mature form of amino acid sequence of Mal3A (SEQ ID NO:3), namely amino acid residues 17-872 of SEQ ID NO:2; or (d) a fragment of (a), (b), (c) having beta-glucosidase activity. In certain embodiments, a variant polypeptide provided having beta-glucosidase activity, which comprises a substitution, a deletion and/or an insertion of one or more amino acid residues (e.g., a few amino acid residues) of Mal3A as shown in SEQ ID NO:2.

In some aspects, a Mal3A polypeptide and/or as it is applied in an enzyme composition or in a method to hydrolyze a lignocellulosic biomass substrate is (a) a polypeptide encoded by a nucleic acid sequence that is at least 75% (e.g., at least 75%, 80%, 85%, 90%, 91%, 92%, 93%, 94%, 95%, 96%, 97%, 98%, 99% or 100%) sequence identity to SEQ ID NO:1, or (b) a polypeptide encoded by a nucleic acid sequence that hybridizes under medium stringency conditions, high stringency conditions or very high stringency conditions to SEQ ID NO:1 or to a subsequence of SEQ ID NO:1 of at least 100 contiguous nucleotides, or to the complementary sequence thereof, wherein the polypeptide has beta-glucosidase activity. In some embodiments, a polynucleotide encoding a Mal3A polypeptide is one that, due to the degeneracy of the genetic code, does not hybridize under medium stringency conditions, high stringency conditions or very high stringency conditions to SEQ ID NO:1 or to a subsequence of SEQ ID NO:1 of at least 100 contiguous nucleotide, but nevertheless encodes a polypeptide having beta-glucosidase activity and comprising an amino acid sequence that is at least 75% (e.g., at least 75%, 80%, 85%, 90%, 91%, 92%, 93%, 94%, 95%, 96%, 97%, 98%, 99% or 100%) identical to that of SEQ ID NO:2 or to the mature beta-glucosidase sequence of SEQ ID NO:3. Nucleic acid sequences encoding a polypeptide having beta-glucosidase activity and comprising an amino acid sequence that is least 75% identical to SEQ ID NO:2 or to SEQ ID NO:3 can be synthetic, and thus not necessarily derived from Melanocarpus albomyces.

In some embodiments, the Mal3A polypeptide or the composition comprising the Mal3A polypeptide has improved beta-glucosidase activity as compared to that of the wild type T. reesei Bgl1 (SEQ ID NO: 4, which is the full length; or SEQ ID NO:5, which is the mature form) or an enzyme composition comprising the T. reesei Bgl1. The comparative beta-glucosidase activity of Mal3A and T. reesei Bgl1 can be determined using any convenient assay, including but not limited to those described in the Examples section below.

For example, in certain embodiments, beta-glucosidase activity can be assessed by determining cellobiase activity. In certain embodiments, cellobiase activity of a Mal3A polypeptide of the compositions and methods herein is at least 5% higher, at least 7% higher, at least 10% higher, at least 15% higher, at least 20% higher, at least 30% higher, at least 40% higher, at least 50% higher, at least 60% higher, at least 70% higher, 80% higher, or at least 90% higher than that of T. reesei Bgl1. In certain embodiments, cellobiase activity is determined using a cellobiose hydrolysis assay, e.g., as described in Example 2B herein.

In other aspects, beta-glucosidase activity can be assessed by determining hydrolysis activity on a lignocellulosic biomass. Thus, in certain embodiments, improved hydrolysis performance of Mal3A polypeptides or compositions comprising Mal3A polypeptides is observable by the production of a greater amount of glucose from a given lignocellulosic biomass substrate, pretreated in a certain way, as compared to the level of glucose produced by T. reesei Bgl1 or an identical enzyme composition comprising T. reesei Bgl1 from the same biomass pretreated the same way, under the same saccharification conditions. For example, the amount of glucose produced by the Mal3A polypeptides or by the enzyme compositions comprising the Mal3A polypeptides is at least 5% greater, at least 10% greater, at least 15% greater, at least 20% greater, at least 25% greater, at least 30% greater, at least 35% greater, at least 40% greater, at least 45% greater, or even at least 50% greater than the amount produced by an equivalent dose (e.g., at the same mg/g ratio of enzyme/glucan in the biomass) of T. reesei Bgl1 or an otherwise identical enzyme composition comprising the T. reesei Bgl1. In certain embodiments, hydrolysis activity on a lignocellulosic biomass is determined using one or more assay as described in Example 2C herein. For example, a Mal3A enzyme as described herein can have a higher temperature optimum and/or higher thermostability as compared to the benchmark T. reesei Bgl1 enzyme, resulting in improved hydrolysis performance on lignocellulosic biomass at elevated temperature as compared to this benchmark (e.g., at 55° C., as described in Example 2C-c).

In some aspects, the improved hydrolysis performance of Mal3A polypeptides or compositions comprising Mal3A polypeptides is observable by the production of an equal or reduced amount of total sugars from a given lignocellulosic biomass substrate pretreated in a certain way, as compared to the level of total sugars produced by equivalent doses/amounts of T. reesei Bgl1 or an otherwise identical enzyme composition comprising T. reesei Bgl1 from the same biomass pretreated the same way, under the same saccharification conditions. For example, the amount of total sugars produced by the Mal3A polypeptides or the enzyme compositions comprising the Mal3A polypeptides is the same or at least 5% less, at least 10% less, at least 15% less, at least 20% less, at least 25% less, at least 30% less, at least 35% less, at least 40% less, at least 45% less, or even at least 50% less than that the amount produced by T. reesei Bgl1 or an otherwise identical enzyme composition comprising T. reesei Bgl1. In certain embodiments, hydrolysis activity on a lignocellulosic biomass is determined using one or more assay as described in Example 2C herein. For example, a Mal3A enzyme as described herein can have a higher temperature optimum and/or higher thermostability as compared to the benchmark T. reesei Bgl1 enzyme, resulting in improved hydrolysis performance on lignocellulosic biomass at elevated temperature as compared to this benchmark (e.g., at 55° C., as described in Example 2C-c).

In further aspects, the improved hydrolysis performance of Mal3A polypeptides and compositions comprising Mal3A polypeptides is observable by an increased amount of glucose and an equal or reduced amount of total sugars produced from hydrolyzing a given lignocellulosic biomass substrate pretreated in a certain way, as compared to the amount of glucose and amount of total sugars produced by T. reesei Bgl1 or an otherwise identical composition comprising T. reesei Bgl1 from the same biomass pretreated the same way under the same saccharification conditions. For example, the amount of glucose produced by the Mal3A polypeptides or the compositions comprising the Mal3A polypeptides is at least 5% greater, at least 10% greater, at least 15% greater, at least 20% greater, at least 25% greater, at least 30% greater, at least 35% greater, at least 40% greater, at least 45% greater, or even at least 50% greater than the amount produced by the T. reesei Bgl1 or by an otherwise identical enzyme composition comprising T. reesei Bgl1, whereas the amount of total sugars produced by the Mal3A polypeptides or the compositions comprising the Mal3A polypeptides is the same or at least 5% less, at least 10% less, at least 15% less, at least 20% less, at least 25% less, at least 30% less, at least 35% less, at least 40% less, at least 45% less, or even at least 50% less than the amount produced by the T. reesei Bgl1 or by an otherwise identical enzyme composition comprising T. reesei Bgl1. In certain embodiments, hydrolysis activity on a lignocellulosic biomass is determined using one or more assay as described in Example 2C herein. For example, a Mal3A enzyme as described herein can have a higher temperature optimum and/or higher thermostability as compared to the benchmark T. reesei Bgl1 enzyme, resulting in improved hydrolysis performance on lignocellulosic biomass at elevated temperature as compared to this benchmark (e.g., at 55° C., as described in Example 2C-c).

Aspects of the present compositions and methods include a composition comprising a recombinant Mal3A polypeptide as detailed above and a lignocellulosic biomass. Suitable lignocellulosic biomass may be, for example, derived from an agricultural crop, a byproduct of a food or feed production, a lignocellulosic waste product, a plant residue, including, for example, a grass residue, or a waste paper or waste paper product. In certain embodiments, the lignocellulosic biomass has been subject to one or more pretreatment steps in order to render xylan, hemicelluloses, cellulose and/or lignin material more accessible or susceptible to enzymes and thus more amenable to enzymatic hydrolysis. A suitable pretreatment method may be, for example, subjecting biomass material to a catalyst comprising a dilute solution of a strong acid and a metal salt in a reactor. See, e.g., U.S. Pat. Nos. 6,660,506 and 6,423,145. Alternatively, a suitable pretreatment may be, for example, a multi-stepped process as described in U.S. Pat. No. 5,536,325. In certain embodiments, the biomass material may be subject to one or more stages of dilute acid hydrolysis using about 0.4% to about 2% of a strong acid, in accordance with the disclosures of U.S. Pat. No. 6,409,841. Further embodiments of pretreatment methods may include those described in, for example, U.S. Pat. No. 5,705,369; in Gould, Biotech. & Bioengr., 26:46-52 (1984); in Texixeira et al., Appl. Biochem & Biotech., 77-79:19-34 (1999); in International Published Patent Application WO2004/081185; or in U.S. Patent Publication No. 20070031918, or International Published Patent Application WO06110901.

The present invention also pertains to isolated polynucleotides encoding polypeptides having beta-glucosidase activity, wherein the isolated polynucleotides are selected from:

(1) a polynucleotide encoding a polypeptide comprising an amino acid sequence having at least 75% (e.g., at least 75%, 80%, 85%, 90%, 91%, 92%, 93%, 94%, 95%, 96%, 97%, 98%, 99%, or 100%) identity to SEQ ID NO:2 or to SEQ ID NO:3;

(2) a polynucleotide having at least 75% (e.g., at least 75%, 80%, 85%, 90%, 91%, 92%, 93%, 94%, 95%, 96%, 97%, 98%, 99%, or 100%) identity to SEQ ID NO:1, or hybridizes under medium stringency conditions, high stringency conditions, or very high stringency conditions to SEQ ID NO:1, or to a complementary sequence thereof.

Aspects of the present compositions and methods include methods of making or producing a Mal3A polypeptide having beta-glucosidase activity, employing an isolated nucleic acid sequence encoding the recombinant polypeptide comprising an amino acid sequence that is at least 75% identical (e.g., at least 75%, 80%, 85%, 90%, 91%, 92%, 93%, 94%, 95%, 96%, 97%, 98%, 99%, or 100%) to that of SEQ ID NO:2, or that of the mature sequence SEQ ID NO:3. In some embodiments, the polypeptide further comprises a native or non-native signal peptide such that the Mal3A polypeptide that is produced is secreted by a host organism, for example, the signal peptide comprises a sequence that is at least 90% identical to SEQ ID NO:7 (the signal sequence of T. reesei Bgl1). In certain embodiments the isolated nucleic acid comprises a sequence that is at least 75% (e.g., at least 75%, 80%, 85%, 90%, 91%, 92%, 93%, 94%, 95%, 96%, 97%, 98%, 99%, or 100%) identical to SEQ ID NO:1. In certain embodiments, the isolated nucleic acid further comprises a nucleic acid sequence encoding a signal peptide sequence. In certain embodiments, the signal peptide sequence may be one selected from SEQ ID NOs: 6-34, 36 and 38. In certain particular embodiments, a nucleic acid sequence encoding the signal peptide sequence of SEQ ID NO:7 is used to express a Mal3A polypeptide in T. reesei.

Aspects of the present compositions and methods include an expression vector comprising the isolated nucleic acid as described above in operable combination with a regulatory sequence.

Aspects of the present compositions and methods include a host cell comprising the expression vector. In certain embodiments, the host cell is a bacterial cell or a fungal cell. In certain embodiments, the host cell comprising the expression vector is an ethanologen microbe capable of metabolizing the soluble sugars produced from a hydrolysis of a lignocellulosic biomass, wherein the hydrolysis is the result of a chemical and/or enzymatic process.

Aspects of the present compositions and methods include a composition comprising the host cell described above and a culture medium. Aspects of the present compositions and methods include a method of producing a Mal3A polypeptide comprising: culturing the host cell described above in a culture medium, under suitable conditions to produce the beta-glucosidase.

Aspects of the present compositions and methods include a composition comprising a Mal3A polypeptide in the supernatant of a culture medium produced in accordance with the methods for producing the beta-glucosidase as described above.

In some aspects, the present invention is related to nucleic acid constructs, recombinant expression vectors, engineered host cells comprising a polynucleotide encoding a polypeptide having beta-glucosidase activity, as described above and herein. In further aspects, the present invention pertains to methods of preparing or producing the beta-glucosidase polypeptides of the invention or compositions comprising such beta-glucosidase polypeptides using the nucleic acid constructs, recombinant expression vectors, and/or engineered host cells. In particular, the present invention is related, for example, to a nucleic acid constructs comprising a suitable signal peptide operably linked to the mature sequence of the beta-glucosidase that is at least 85% identical to SEQ ID NO:2 or to the mature sequence of SEQ ID NO:3, or is encoded by a polynucleotide that is at least 85% identical to SEQ ID NO:1, an isolated polynucleotide, a nucleic acid construct, a recombinant expression vector, or an engineered host cell comprising such a nucleic acid construct. In some embodiments, the signal peptide and beta-glucosidase sequences are derived from different microorganisms.

Also provided is an expression vector comprising the isolated nucleic acid in operable combination with a regulatory sequence. Additionally, a host cell is provided comprising the expression vector. In still further embodiments, a composition is provided, which comprises the host cell and a culture medium.

In some embodiments, the host cell is a bacterial cell or a fungal cell. In certain embodiments, the host cell is an ethanologen microbe, which is capable of metabolizing the soluble sugars produced from hydrolyzing a lignocellulosic biomass substrate, wherein the hydrolyzing can be through a chemical hydrolysis or enzymatic hydrolysis or a combination of these processes, but is also capable of expression of heterologous enzymes. In some embodiments, the host cell is a Saccharomyces cerevisiae or a Zymomonas mobilis cell, which are not only capable of expressing a heterologous polypeptide such as a Mal3A polypeptide of the invention, but also capable of fermenting sugars into ethanol and/or downstream products. In certain particular embodiments, the Saccharomyces cerevisiae cell or Zymomonas mobilis cell, which expresses the beta-glucosidase, is capable of fermenting the sugars produced from a lignocellulosic biomass by an enzyme composition comprising one or more beta-glucosidases.

Furthermore, no fermenting or ethanologen microorganism capable of converting cellulosic sugars obtained from enzymatic hydrolysis of lignocellulosic biomass has been engineered to express a beta-glucosidase from Melanocarpus albomyces, such as a Mal3A polypeptide as described herein. Expression of beta-glucosidases in ethanologen microorganisms provides an important opportunity to further liberating D-glucose from the remaining cellobiose that are not completely converted by enzymatic saccharification, where the D-glucose thus produced can be immediately consumed or fermented just in time by the ethanologen.

The enzyme composition comprising one or more beta-glucosidases may comprise the same beta-glucosidase or may comprise one or more different beta-glucosidases. In certain embodiments, the enzyme composition comprising one or more beta-glucosidases may be an enzyme mixture produced by an engineered host cell, which may be a bacterial or a fungal cell. When a Saccharomyces cerevisiae or a Zymomonas mobilis cell expressing the Mal3A polypeptide of the present disclosure, the Mal3A polypeptide may be expressed but not secreted. Accordingly the cellobiose must be introduced or “transported” into such a host cell in order for the beta-glucosidase Mal3A polypeptide to catalyze the liberation of D-glucose. Therefore in certain embodiments, the Saccharomyces cerevisiae or a Zymomonas mobilis cell are transformed with a cellobiose transporter gene in addition to one that encodes the Mal3A polypeptide. A cellobiose transporter and a beta-glucosidase have been expressed in Saccharomyces cerevisiae such that the resulting microbe is capable of fermenting cellobiose, for example, in Ha et al., PNAS, 108(2):504-509 (2011). Another cellobiose transporter has been expressed in a Pichia yeast, for example in published U.S. Patent Application No. 20110262983. A cellobiose transporter has been introduced into an E. coli, for example, in Sekar et al., Applied Environmental Microbiology, 78(5):1611-1614 (2012).

In further embodiments, the Mal3A polypeptide is heterologously expressed by a host cell. For example, the Mal3A polypeptide is expressed by an engineered microorganism that is not Melanocarpus albomyces. In some embodiments, the Mal3A polypeptide is co-expressed with one or more different cellulase genes. In some embodiments, the Mal3A polypeptide is co-expressed with one or more hemicellulase genes.

In some aspects, compositions comprising the recombinant Mal3A polypeptides of the preceding paragraphs and methods of preparing such compositions are provided. In some embodiments, the composition further comprises one or more other cellulases, whereby the one or more other cellulases are co-expressed by a host cell with the Mal3A polypeptide. For example, the one or more other cellulases can be selected from no or one or more other beta-glucosidases, one or more cellobiohydrolases, and/or one or more endoglucanases. Such other beta-glucosidases, cellobiohydrolases and/or endoglucanases, if present, can be co-expressed with the Mal3A polypeptide by a single host cell. At least two of the two or more cellulases may be heterologous to each other or derived from different organisms. For example, the composition may comprise two beta-glucosidases, with the first one being a Mal3A polypeptide, and the second beta-glucosidase being not derived from a Melanocarpus albomyces strain. For example, the composition may comprise at least one cellobiohydrolase, one endoglucanase, or one beta-glucosidase that is not derived from Melanocarpus albomyces. In some embodiments, one or more of the cellulases are endogenous to the host cell, but are overexpressed or expressed at a level that is different from that would otherwise be naturally-occurring in the host cell. For example, one or more of the cellulases may be a T. reesei CBH1 and/or CBH2, which are native to a T. reesei host cell, but either or both CBH1 and CBH2 are overexpressed or underexpressed when they are co-expressed in the T. reesei host cell with a Mal3A polypeptide.

In certain embodiments, the composition comprising the recombinant Mal3A polypeptide may further comprise one or more hemicellulases, whereby the one or more hemicellulases are co-expressed by a host cell with the Mal3A polypeptide. For example, the one or more hemicellulases can be selected from one or more xylanase, one or more beta-xylosidases, and/or one or more α-L-arabinofuranosidases. Such other xylanases, beta-xylosidases and L-arabinofuranosidases, if present, can be co-expressed with the Mal3A polypeptide by a single host cell. In some embodiments, the composition may comprise at least one beta-xylosidase, xylanase or arabinofuranosidase that is not derived from Melanocarpus albomyces.

In further aspects, the composition comprising the recombinant Mal3A polypeptide may further comprise one or more other celluases and one or more hemicelluases, whereby the one or more cellulases and/or one or more hemicellulases are co-expressed by a host cell with the Mal3A polypeptide. For example, a Mal3A polypeptide may be co-expressed with one or more other beta-glucosidases, one or more cellobiohydrolases, one or more endoglucanases, one or more endo-xylanases, one or more beta-xylosidases, and one or more α-L-arabinofuranosidases, in addition to other non-cellulase non-hemicellulase enzymes or proteins in the same host cell. Aspects of the present compositions and methods accordingly include a composition comprising the host cell described above co-expressing a number of enzymes in addition to the Mal3A polypeptide and a culture medium. Aspects of the present compositions and methods accordingly include a method of producing a Mal3A-containing enzyme composition comprising: culturing the host cell, which co-expresses a number of enzymes as described above with the Mal3A polypeptide in a culture medium, under suitable conditions to produce the Mal3A and the other enzymes. Also provided are compositions that comprise the Mal3A polypeptide and the other enzymes produced in accordance with the methods herein in supernatant of the culture medium. Such supernatant of the culture medium can be used as is, with minimum or no post-production processing, which may typically include filtration to remove cell debris, cell-kill procedures, and/or ultrafiltration or other steps to enrich or concentrate the enzymes therein. Such supernatants are called “whole broths” or “whole cellulase broths” herein.

In further aspects, the present invention pertains to a method of applying or using the composition as described above under conditions suitable for degrading or converting a cellulosic material and for producing a substance from a cellulosic material.

In a further aspect, methods for degrading or converting a cellulosic material into fermentable sugars are provided, comprising: contacting the cellulosic material, preferably having already been subject to one or more pretreatment steps, with the Mal3A polypeptides or the compositions comprising such polypeptides of one of the preceding paragraphs to yield fermentable sugars.

These and other aspects of Mal3A compositions and methods will be apparent from the following description.

### DESCRIPTION OF THE DRAWINGS

FIG. 1 depicts a map of the pENTR/D-TOPO-Bgl1(943/942) vector.

FIG. 2 depicts a map of the pTrex3g 943/942 construct for expression in a Trichoderma reesei host cell.

FIG. 3 depicts a map of Mal bglA (also called Mal3A) in the pENTR/D-TOPO vector.

FIG. 4 depicts a map of Mal bglA with introns (Mal3A) in the pTTT pyr2 vector.

FIG. 5 depicts a yeast shuttle vector pSC11 construct comprising a Mal3A gene optimized and synthesized for expression of the Mal3A polypeptide in a Saccharomyces cerevisiae ethanologen.

FIG. 6 depicts a Zymomonas mobilis integration vector pZC11 comprising a Mal3A gene optimized and synthesized for expression of the Mal3A polypeptide in a Zymomonas mobilis ethanologen.

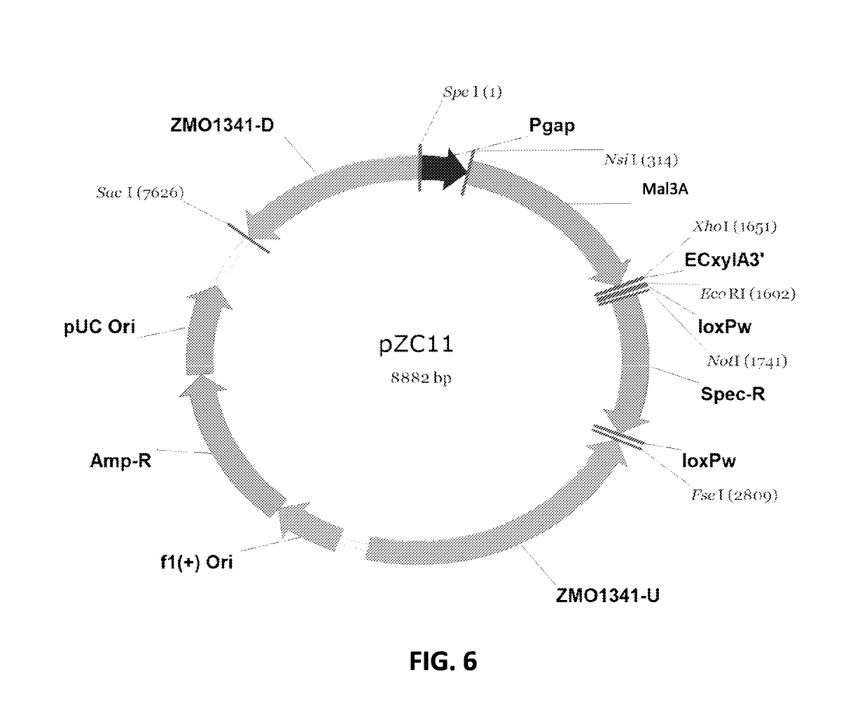

### DETAILED DESCRIPTION

### I. Overview

Described herein are compositions and methods relating to a recombinant beta-glucosidase Mal3A belonging to glycosyl hydrolase family 3 from Melanocarpus albomyces. The present compositions and methods are based, in part, on the observations that recombinant Mal3A polypeptides have higher cellulase activities and are more robust as a component of an enzyme composition when the composition is used to hydrolyze a lignocellulosic biomass material or feedstock than, for example, a known benchmark high fidelity beta-glucosidase Bgl1 of T. reesei. These features of Mal3A polypeptides make them, or variants thereof, suitable for use in numerous processes, including, for example, in the conversion or hydrolysis of a lignocellulosic biomass feedstock.

Before the present compositions and methods are described in greater detail, it is to be understood that the present compositions and methods are not limited to particular embodiments described, as such may, of course, vary. It is also to be understood that the terminology used herein is for the purpose of describing particular embodiments only, and is not intended to be limiting, since the scope of the present compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present compositions and methods are not entitled to antedate such publication by virtue of prior invention. Further, the dates of publication provided may be different from the actual publication dates which may need to be independently confirmed.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

### II. Definitions

“Beta-glucosidase” refers to a beta-D-glucoside glucohydrolase of E.C. 3.2.1.21. The term “beta-glucosidase activity” therefore refers the capacity of catalyzing the hydrolysis of cellobiose, cello-oligosaccharides, and other glucosides to release β-D-glucose. Beta-glucosidase activity may be determined using a cellobiase assay, for example, which measures the capacity of the enzyme to catalyze the hydrolysis of a cellobiose substrate to yield β-D-glucose, see, e.g., the assay described in Example 2C of the present disclosure.

As used herein, “Mal3A” or “a Mal3A polypeptide” refers to a beta-glucosidase belonging to glycosyl hydrolase family 3 (e.g., a recombinant beta-glucosidase) derived from Melanocarpus albomyces (and variants thereof), that has improved performance when compared to a benchmark beta-glucosidase, e.g., the wild type T. reesei Bgl1 polypeptide having or comprising the amino acid sequence of SEQ ID NO:5, in at least one beta-glucosidase assay (e.g., cellobiase activity assay and/or a lignocellulosic biomass substrate hydrolysis assay). According to aspects of the present compositions and methods, Mal3A polypeptides include those comprising the amino acid sequence depicted in SEQ ID NO:2 (full length sequence) or SEQ ID NO:3 (mature form), as well as derivative or variant polypeptides having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:2 or to SEQ ID NO:3, or to a fragment of at least 100 residues in length of SEQ ID NO:2 or SEQ ID NO:3. Mal3A polypeptides according to aspects of the present compositions and methods described herein can be isolated or purified (as defined below).

“Glycosyl hydrolase Family 3” or “GH3” refers to polypeptides falling within the definition of glycosyl hydrolase family 3 according to the classification by Henrissat, Biochem. J. 280:309-316 (1991), and by Henrissat & Cairoch, Biochem. J., 316:695-696 (1996).

By “purified” or “isolated” or “enriched” is meant that a biomolecule (e.g., a polypeptide or polynucleotide) is altered from its natural state by virtue of separating it from some or all of the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition (e.g., purified Mal3A) which provide additional benefits, for example, activating agents, anti-inhibition agents, desirable ions, compounds to control pH or other enzymes or chemicals.

As used herein, “microorganism” refers to a bacterium, a fungus, a virus, a protozoan, and other microbes or microscopic organisms.

As used herein, a “derivative” or “variant” of a polypeptide means a polypeptide, which is derived from a precursor polypeptide (e.g., the native polypeptide) by addition of one or more amino acids to either or both the C- and N-terminal ends, substitution of one or more amino acids at one or a number of different sites in the amino acid sequence, deletion of one or more amino acids at either or both ends of the polypeptide or at one or more sites in the amino acid sequence, or insertion of one or more amino acids at one or more sites in the amino acid sequence. The preparation of a Mal3A derivative or variant may be achieved in any convenient manner, e.g., by modifying a DNA sequence which encodes the native polypeptides, transformation of that DNA sequence into a suitable host, and expression of the modified DNA sequence to form the derivative/variant Mal3A. Derivatives or variants further include Mal3A polypeptides that are chemically modified, e.g., glycosylation or otherwise changing a characteristic of the Mal3A polypeptide. While derivatives and variants of Mal3A are encompassed by the present compositions and methods, such derivates and variants will display improved beta-glucosidase activity when compared to that of the wild type T. reesei Bgl1 of SEQ ID NO:4 (full length) or SEQ ID NO:5 (mature form), under the same lignocellulosic biomass substrate hydrolysis conditions.

In certain aspects, a Mal3A polypeptide of the compositions and methods herein may also encompasses functional fragment of a polypeptide or a polypeptide fragment having beta-glucosidase activity, which is derived from a parent polypeptide, which may be the full length polypeptide comprising or consisting of SEQ ID NO:2, or the mature sequence comprising or consisting SEQ ID NO:3. The functional polypeptide may have been truncated either in the N-terminal region, or the C-terminal region, or in both regions to generate a fragment of the parent polypeptide. For the purpose of the present disclosure, a functional fragment must have at least 20%, more preferably at least 30%, 40%, 50%, or preferably, at least 60%, 70%, 80%, or even more preferably at least 90% of the beta-glucosidase activity of that of the parent polypeptide.

In certain aspects, a Mal3A derivative/variant will have anywhere from 75% to 99% (or more) amino acid sequence identity to the amino acid sequence of SEQ ID NO:2, or to the mature sequence SEQ ID NO:3, e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity to the amino acid sequence of SEQ ID NO:2 or to the mature sequence SEQ ID NO:3. In some embodiments, amino acid substitutions are “conservative amino acid substitutions” using L-amino acids, wherein one amino acid is replaced by another biologically similar amino acid. Conservative amino acid substitutions are those that preserve the general charge, hydrophobicity/hydrophilicity, and/or steric bulk of the amino acid being substituted. Examples of conservative substitutions are those among the following groups: Gly/Ala, Val/Ile/Leu, Lys/Arg, Asn/Gln, Glu/Asp, Ser/Cys/Thr, and Phe/Trp/Tyr. A derivative may, for example, differ by as few as 1 to 10 amino acid residues, such as 6-10, as few as 5, as few as 4, 3, 2, or even 1 amino acid residue. In some embodiments, a Mal3A derivative may have an N-terminal and/or C-terminal deletion, where the Mal3A derivative excluding the deleted terminal portion(s) is identical to a contiguous sub-region in SEQ ID NO: 2 or SEQ ID NO:3.

As used herein, “percent (%) sequence identity” with respect to the amino acid or nucleotide sequences identified herein is defined as the percentage of amino acid residues or nucleotides in a candidate sequence that are identical with the amino acid residues or nucleotides in a Mal3A sequence, after aligning the sequences and introducing gaps, if necessary, to achieve the maximum percent sequence identity, and not considering any conservative substitutions as part of the sequence identity.

By “homologue” (or “homolog”) shall mean an entity having a specified degree of identity with the subject amino acid sequences and the subject nucleotide sequences. A homologous sequence is taken to include an amino acid sequence that is at least 75%, 80%, 85%, 86%, 87%, 88%, 89%, 90%, 91%, 92%, 93%, 94%, 95%, 96%, 97%, 98% or even 99% identical to the subject sequence, using conventional sequence alignment tools (e.g., Clustal, BLAST, and the like). Typically, homologues will include the same active site residues as the subject amino acid sequence, unless otherwise specified.

Methods for performing sequence alignment and determining sequence identity are known to the skilled artisan, may be performed without undue experimentation, and calculations of identity values may be obtained with definiteness. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Protein Sequence and Structure 5:Suppl. 3 (National Biomedical Research Foundation, Washington, D.C.). A number of algorithms are available for aligning sequences and determining sequence identity and include, for example, the homology alignment algorithm of Needleman et al. (1970) J. Mol. Biol. 48:443; the local homology algorithm of Smith et al. (1981) Adv. Appl. Math. 2:482; the search for similarity method of Pearson et al. (1988) Proc. Natl. Acad. Sci. 85:2444; the Smith-Waterman algorithm (Meth. Mol. Biol. 70:173-187 (1997); and BLASTP, BLASTN, and BLASTX algorithms (see Altschul et al. (1990) J. Mol. Biol. 215:403-410).

Computerized programs using these algorithms are also available, and include, but are not limited to: ALIGN or Megalign (DNASTAR) software, or WU-BLAST-2 (Altschul et al., Meth. Enzym., 266:460-480 (1996)); or GAP, BESTFIT, BLAST, FASTA, and TFASTA, available in the Genetics Computing Group (GCG) package, Version 8, Madison, Wis., USA; and CLUSTAL in the PC/Gene program by Intelligenetics, Mountain View, Calif. Those skilled in the art can determine appropriate parameters for measuring alignment, including algorithms needed to achieve maximal alignment over the length of the sequences being compared. Preferably, the sequence identity is determined using the default parameters determined by the program. Specifically, sequence identity can determined by using Clustal W (Thompson J. D. et al. (1994) Nucleic Acids Res. 22:4673-4680) with default parameters, i.e.:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

As used herein, “expression vector” means a DNA construct including a DNA sequence that encodes one or more specified polypeptides that is operably linked to a suitable control sequence capable of affecting the expression of the one or more polypeptides in a suitable host. Such control sequences may include a promoter to affect transcription, an optional operator sequence to control transcription, a sequence encoding suitable ribosome-binding sites on the mRNA, and sequences which control termination of transcription and translation. Different cell types may be used with different expression vectors. An exemplary promoter for vectors used in Bacillus subtilis is the AprE promoter; an exemplary promoter used in Streptomyces lividans is the A4 promoter (from Aspergillus niger); an exemplary promoter used in E. coli is the Lac promoter, an exemplary promoter used in Saccharomyces cerevisiae is PGK1, an exemplary promoter used in Aspergillus niger is glaA, and an exemplary promoter for T. reesei is cbhI. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the present compositions and methods are intended to include other forms of expression vectors which serve equivalent functions and which are, or become, known in the art. Thus, a wide variety of host/expression vector combinations may be employed in expressing the DNA sequences described herein. Useful expression vectors, for example, may consist of segments of chromosomal, non-chromosomal and synthetic DNA sequences such as various known derivatives of SV40 and known bacterial plasmids, e.g., plasmids from E. coli including col E1, pCR1, pBR322, pMb9, pUC 19 and their derivatives, wider host range plasmids, e.g., RP4, phage DNAs e.g., the numerous derivatives of phage λ, e.g., NM989, and other DNA phages, e.g., M13 and filamentous single stranded DNA phages, yeast plasmids such as the 2μ plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors of the present compositions and methods are known in the art and are described generally in, for example, Sambrook et al., Molecular Cloning: A Laboratory Manual, Second Edition, Cold Spring Harbor Press (1989). Often, such expression vectors including the DNA sequences described herein are transformed into a unicellular host by direct insertion into the genome of a particular species through an integration event (see e.g., Bennett & Lasure, More Gene Manipulations in Fungi, Academic Press, San Diego, pp. 70-76 (1991) and articles cited therein describing targeted genomic insertion in fungal hosts).

As used herein, “host strain” or “host cell” means a suitable host for an expression vector including DNA according to the present compositions and methods. Host cells useful in the present compositions and methods are generally prokaryotic or eukaryotic hosts, including any transformable microorganism in which expression of a desired polypeptide/enzyme (or multiple polypeptides/enzymes) can be achieved. Specifically, host strains may be Bacillus subtilis, Streptomyces lividans, Escherichia coli, Trichoderma reesei, Saccharomyces cerevisiae or Aspergillus niger. In certain embodiments, the host cell may be an ethanologen microbe, which may be, for example, a yeast such as Saccharomyces cerevisiae or a bacterium ethanologen such as a Zymomonas mobilis. When a Saccharomyces cerevisiae or Zymomonas mobilis is used as the host cell, and if the beta-glucosidase gene is not made to secret from host cell but is expressed intracellularly, a cellobiose transporter gene can be introduced into the host cell in order to allow the intracellularly expressed beta-glucosidase to act upon the cellobiose substrate and liberate glucose, which will then be metabolized subsequently or immediately by the microorganisms and converted into ethanol.

Host cells are transformed or transfected with vectors constructed using recombinant DNA techniques. Such transformed host cells may be capable of one or both of replicating the vectors encoding Mal3A (and its derivatives or variants (mutants)) and expressing the desired peptide product. In certain embodiments according to the present compositions and methods, “host cell” means both the cells and protoplasts created from the cells of Trichoderma sp.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

The term “heterologous” with reference to a polynucleotide or polypeptide refers to a polynucleotide or polypeptide that does not naturally occur in a host cell.

The term “endogenous” with reference to a polynucleotide or polypeptide refers to a polynucleotide or polypeptide that occurs naturally in the host cell.

The term “expression” refers to the process by which a polypeptide is produced based on a nucleic acid sequence. The process includes both transcription and translation.

The term “recombinant,” when used in reference to a biological component or composition (e.g., a cell, nucleic acid, polypeptide/enzyme, vector, etc.) indicates that the biological component or composition is in a state that is not found in nature. In other words, the biological component or composition has been modified by human intervention from its natural state. For example, a recombinant cell encompass a cell that expresses one or more genes that are not found in its native parent (i.e., non-recombinant) cell, a cell that expresses one or more native genes in an amount that is different than its native parent cell, and/or a cell that expresses one or more native genes under different conditions than its native parent cell. Recombinant nucleic acids may differ from a native sequence by one or more nucleotides, be operably linked to heterologous sequences (e.g., a heterologous promoter, a sequence encoding a non-native or variant signal sequence, etc.), be devoid of intronic sequences, and/or be in an isolated form. Recombinant polypeptides/enzymes may differ from a native sequence by one or more amino acids, may be fused with heterologous sequences, may be truncated or have internal deletions of amino acids, may be expressed in a manner not found in a native cell (e.g., from a recombinant cell that over-expresses the polypeptide due to the presence in the cell of an expression vector encoding the polypeptide), and/or be in an isolated form. It is emphasized that in some embodiments, a recombinant polynucleotide or polypeptide/enzyme has a sequence that is identical to its wild-type counterpart but is in a non-native form (e.g., in an isolated or enriched form).

As used herein, “signal sequence” means a sequence of amino acids bound to the N-terminal portion of a polypeptide which facilitates the secretion of the mature form of the polypeptide outside of the cell. This definition of a signal sequence is a functional one. The mature form of the extracellular polypeptide lacks the signal sequence which is cleaved off during the secretion process. While the native signal sequence of Mal3A may be employed (SEQ ID NO:6) in aspects of the present compositions and methods, other non-native signal sequences may be employed (e.g., SEQ ID NO: 7). The term “mature,” when referring to a polypeptide herein, is meant a polypeptide in its final form(s) following translation and any post-translational modifications. For example, the Mal3A polypeptides of the invention has one or more mature forms, at least one of which has the amino acid sequence of SEQ ID NO:3.

The beta-glucosidase polypeptides of the invention may be referred to as “precursor,” “immature,” or “full-length,” in which case they include a signal sequence, or may be referred to as “mature,” in which case they lack a signal sequence. Mature forms of the polypeptides are generally the most useful. Unless otherwise noted, the amino acid residue numbering used herein refers to the mature forms of the respective amylase polypeptides. The beta-glucosidase polypeptides of the invention may also be truncated to remove the N or C-termini, so long as the resulting polypeptides retain beta-glucosidase activity.

The beta-glucosidase polypeptides of the invention may also be a “chimeric” or “hybrid” polypeptide, in that it includes at least a portion of a first beta-glucosidase polypeptide, and at least a portion of a second beta-glucosidase polypeptide (such chimeric beta-glucosidase polypeptides may, for example, be derived from the first and second beta-glucosidases using known technologies involving the swapping of domains on each of the beta-glucosidases). The present beta-glucosidase polypeptides may further include heterologous signal sequence, an epitope to allow tracking or purification, or the like. When the term “heterologous” is used to refer to a signal sequence used to express a polypeptide of interest, it is meant that the signal sequence is, for example, derived from a different microorganism as the polypeptide of interest. Examples of suitable heterologous signal sequences for expressing the Mal3A polypeptides herein, may be, for example, those from T. reesei (e.g., SEQ ID NO:7). Signal sequences as set forth in SEQ ID NOs: 8-34, 36 and 38 may also be used.

As used herein, “functionally attached” or “operably linked” means that a regulatory region or functional domain having a known or desired activity, such as a promoter, terminator, signal sequence or enhancer region, is attached to or linked to a target (e.g., a gene or polypeptide) in such a manner as to allow the regulatory region or functional domain to control the expression, secretion or function of that target according to its known or desired activity.

As used herein, the terms “polypeptide” and “enzyme” are used interchangeably to refer to polymers of any length comprising amino acid residues linked by peptide bonds. The conventional one-letter or three-letter codes for amino acid residues are used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, “wild-type” and “native” genes, enzymes, or strains, are those found in nature.

The terms “wild-type,” “parental,” or “reference,” with respect to a polypeptide, refer to a naturally-occurring polypeptide that does not include a man-made substitution, insertion, or deletion at one or more amino acid positions. Similarly, the term “wild-type,” “parental,” or “reference,” with respect to a polynucleotide, refers to a naturally-occurring polynucleotide that does not include a man-made nucleoside change. However, a polynucleotide encoding a wild-type, parental, or reference polypeptide is not limited to a naturally-occurring polynucleotide, but rather encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues. They may be defined by their level of primary amino acid sequence homology/identity with a parent polypeptide. Suitably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity to a parent polypeptide.

As used herein, a “variant polynucleotide”, for example one that encodes a variant polypeptide, has a specified degree of homology/identity with a parent polynucleotide, or hybridized under stringent conditions to a parent polynucleotide or the complement thereof. Suitably, a variant polynucleotide has at least 80%, at least 85%, at least 90%, at least 91%, at least 92%, at least 93%, at least 94%, at least 95%, at least 96%, at least 97%, at least 98%, or even at least 99% nucleotide sequence identity to a parent polynucleotide or to a complement of the parent polynucleotide. Methods for determining percent identity are known in the art and described above. It is noted here that due to the degeneracy of the genetic code, a variant polynucleotide may encode a non-variant (e.g., a parental) polypeptide, for example when generating a codon optimized polynucleotide for a polypeptide.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm−5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Alternatively, or in addition, hybridization conditions can be based upon the salt or ionic strength conditions of hybridization, and/or upon one or more stringency washes, e.g. 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g. relatively low salt and/or high temperature conditions are used).

As used herein, the term “hybridization” refers to the process by which a strand of nucleic acid joins with a complementary strand through base pairing, as known in the art. More specifically, “hybridization” refers to the process by which one strand of nucleic acid forms a duplex with, i.e., base pairs with, a complementary strand, as occurs during blot hybridization techniques and PCR techniques. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while intermediate or low stringency hybridization can be used to identify or detect polynucleotide sequence homologs.

Intermediate and high stringency hybridization conditions are well known in the art. For example, intermediate stringency hybridizations may be carried out with an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/mL denatured sheared salmon sperm DNA, followed by washing the filters in 1×SSC at about 37-50° C. High stringency hybridization conditions may be hybridization at 65° C. and 0.1×SSC (where 1×SSC=0.15 M NaCl, 0.015 M Na citrate, pH 7.0). Alternatively, high stringency hybridization conditions can be carried out at about 42° C. in 50% formamide, 5×SSC, 5×Denhardt's solution, 0.5% SDS and 100 μg/mL denatured carrier DNA followed by washing two times in 2×SSC and 0.5% SDS at room temperature and two additional times in 0.1×SSC and 0.5% SDS at 42° C. And very high stringent hybridization conditions may be hybridization at 68° C. and 0.1×SSC. Those of skill in the art know how to adjust the temperature, ionic strength, etc. as necessary to accommodate factors such as probe length and the like.

A nucleic acid encoding a variant beta-glucosidase may have a Tₘ reduced by 1° C.-3° C. or more compared to a duplex formed between the nucleotide of SEQ ID NO: 1 and its identical complement.

The phrase “substantially similar” or “substantially identical,” in the context of at least two nucleic acids or polypeptides, means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

The term “selective marker” or “selectable marker,” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element,” refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

An “ethanologenic microorganism” refers to a microorganism with the ability to convert a sugar or oligosaccharide to ethanol.

The term “thermostable” or “thermostability” in reference to a Mal3A enzyme means that the Mal3A enzyme retains a greater fraction of enzymatic activity after a period of incubation at an elevated temperature relative to a benchmark T. reesei Bgl1 enzyme.

By “high thermal activity profile” or “high optimal temperature” in reference to a Mal3A enzyme means that the temperature range in which the Mal3A enzyme has enzymatic activity and/or the optimal temperature for enzyme activity is higher than the temperature range or temperature optimum of the benchmark T. reesei Bgl1 enzyme.

Other technical and scientific terms have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, NY 1991).

### III. Beta-Glucosidase Polypeptides, Polynucleotides, Vectors, and Host Cells

A. Mal3A Polypeptides

In one aspect, the present compositions and methods provide a recombinant Mal3A beta-glucosidase polypeptide, fragments thereof, or variants thereof having beta-glucosidase activity. An example of a recombinant beta-glucosidase polypeptide was isolated from Melanocarpus albomyces. The mature Mal3A polypeptide has the amino acid sequence set forth as SEQ ID NO:3. Similar, or substantially similar, Mal3A polypeptides may occur in nature, e.g., in other strains or isolates of Melanocarpus albomyces. These and other recombinant Mal3A polypeptides are encompassed by the present compositions and methods.

In some embodiments, the recombinant Mal3A polypeptide is a variant Mal3A polypeptide having a specified degree of amino acid sequence identity to the exemplified Mal3A polypeptide, e.g., at least 75%, 80%, 85%, 90%, 91%, 92%, 93%, 94%, 95%, 96%, 97%, 98%, or even at least 99% sequence identity to the amino acid sequence of SEQ ID NO:2 or to the mature sequence SEQ ID NO:3. Sequence identity can be determined by amino acid sequence alignment, e.g., using a program such as BLAST, ALIGN, or CLUSTAL, as described herein. In certain embodiments, variant recombinant Mal3A polypeptide comprises a few amino acid residue substitutions (e.g., 1, 2, 3, 4, 5, 6, 7, 8, 9, 10, or more amino acid residue substitutions).

In certain embodiments, the recombinant Mal3A polypeptides are produced recombinantly, in a microorganism, for example, in a bacterial or fungal host organism, while in others the Mal3A polypeptides are produced synthetically, or are purified from a native source (e.g., Melanocarpus albomyces).

In certain embodiments, the recombinant Mal3A polypeptide includes substitutions that do not substantially affect the structure and/or function of the polypeptide. Examples of these substitutions are conservative mutations, as summarized in Table I.

TABLE I
Amino Acid Substitutions

| Original Residue | Code | Acceptable Substitutions |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, |
| | | D-Leu, Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyro sine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a polynucleotide encoding a recombinant Mal3A polypeptide, and then expressing the variant polypeptide in an organism. Substitutions involving non-naturally occurring amino acids or chemical modifications to amino acids are generally made by chemically modifying a Mal3A polypeptide after it has been synthesized by an organism.

In some embodiments, variant recombinant Mal3A polypeptides are substantially identical to SEQ ID NO:2 or SEQ ID NO:3, meaning that they do not include amino acid substitutions, insertions, or deletions that significantly affect the structure, function, or expression of the polypeptide. Such variant recombinant Mal3A polypeptides will include those designed to circumvent the present description. In some embodiments, variant recombinant Mal3A polypeptides, compositions and methods comprising these variants are not substantially identical to SEQ ID NO:2 or SEQ ID NO:3, but rather include amino acid substitutions, insertions, or deletions that affect, in certain circumstances, substantially, the structure, function, or expression of the polypeptide herein such that improved characteristics, including, e.g., improved specific activity to hydrolyze a lignocellulosic substrate, improved expression in a desirable host organism, improved thermostability, pH stability, etc., as compared to that of a polypeptide of SEQ ID NO:2 or SEQ ID NO:3 can be achieved. In certain embodiments, variant recombinant Mal3A polypeptide comprises a few amino acid residue substitutions (e.g., 1, 2, 3, 4, 5, 6, 7, 8, 9, 10, or more amino acid residue substitutions).

In some embodiments, the recombinant Mal3A polypeptide (including a variant thereof) has beta-glucosidase activity. Beta-glucosidase activity can be determined and measured using the assays described herein, for example, those described in Example 2, or by other assays known in the art.

Recombinant Mal3A polypeptides include fragments of “full-length” Mal3A polypeptides that retain beta-glucosidase activity. Preferably those functional fragments (i.e., fragments that retain beta-glucosidase activity) are at least 100 amino acid residues in length (e.g., at least 100 amino acid residues, at least 120 amino acid residues, at least 140 amino acid residues, at least 160 amino acid residues, at least 180 amino acid residues, at least 200 amino acid residues, at least 220 amino acid residues, at least 240 amino acid residues, at least 260 amino acid residues, at least 280 amino acid residues, at least 300 amino acid residues, at least 320 amino acid residues, or at least 350 amino acid residues in length or longer). Such fragments suitably retain the active site of the full-length precursor polypeptides or full length mature polypeptides but may have deletions of non-critical amino acid residues. The activity of fragments can be readily determined using the assays described herein, for example those described in Example 2, or by other assays known in the art.

In some embodiments, the Mal3A amino acid sequences and derivatives are produced as an N- and/or C-terminal fusion protein, for example, to aid in extraction, detection and/or purification and/or to add functional properties to the Mal3A polypeptides. Examples of fusion protein partners include, but are not limited to, glutathione-S-transferase (GST), 6×His, GAL4 (DNA binding and/or transcriptional activation domains), FLAG-, MYC-tags or other tags known to those skilled in the art. In some embodiments, a proteolytic cleavage site is provided between the fusion protein partner and the polypeptide sequence of interest to allow removal of fusion sequences. Suitably, the fusion protein does not hinder the activity of the recombinant Mal3A polypeptide. In some embodiments, the recombinant Mal3A polypeptide is fused to a functional domain including a leader peptide, propeptide, binding domain and/or catalytic domain. Fusion proteins are optionally linked to the recombinant Mal3A polypeptide through a linker sequence that joins the Mal3A polypeptide and the fusion domain without significantly affecting the properties of either component. The linker optionally contributes functionally to the intended application.

The present disclosure provides host cells that are engineered to express one or more Mal3A polypeptides of the disclosure. Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus licheniformis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

Methods of transforming nucleic acids into these organisms are known in the art. For example, a suitable procedure for transforming Aspergillus host cells is described in EP 238023.

In some embodiments, the recombinant Mal3A polypeptide is fused to a signal peptide to, for example, facilitate extracellular secretion of the recombinant Mal3A polypeptide. For example, in certain embodiments, the signal peptide comprises a sequence selected from SEQ ID NOs: 6-34, 36 and 38. Recombinant polynucleotides encoding such signal peptides/Mal3A fusions can be made using standard molecular genetics techniques (i.e., to place a polynucleotide sequence encoding the desired signal peptide in operable linkage with a polynucleotide encoding a Mal3A polypeptide). In particular embodiments, the recombinant Mal3A polypeptide is expressed in a heterologous organism as a secreted polypeptide. The compositions and methods herein thus encompass methods for expressing a Mal3A polypeptide as a secreted polypeptide in a heterologous organism. In some embodiments the recombinant Mal3A polypeptide is expressed in a heterologous organism intracellularly, for example, when the heterologous organism is an ethanologen microbe such as a Saccharomyces cerevisiae or a Zymomonas mobilis. In those cases, a cellobiose transporter gene can be introduced into the organism using genetic engineering tools, in order for the Mal3A polypeptide to act on the cellobiose substrate inside the organism to convert cellobiose into D-glucose, which is then metabolized or converted by the organism into ethanol.

The disclosure also provides expression cassettes and/or vectors comprising the above-described nucleic acids. Suitably, the nucleic acid encoding a Mal3A polypeptide of the disclosure is operably linked to a promoter. Promoters are well known in the art. Any promoter that functions in the host cell can be used for expression of a beta-glucosidase and/or any of the other nucleic acids of the present disclosure. Initiation control regions or promoters, which are useful to drive expression of a beta-glucosidase nucleic acids and/or any of the other nucleic acids of the present disclosure in various host cells are numerous and familiar to those skilled in the art (see, for example, WO 2004/033646 and references cited therein). Virtually any promoter capable of driving these nucleic acids can be used.

Specifically, where recombinant expression in a filamentous fungal host is desired, the promoter can be a filamentous fungal promoter. The nucleic acids can be, for example, under the control of heterologous promoters. The nucleic acids can also be expressed under the control of constitutive or inducible promoters. Examples of promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or beta-glucosidase promoter. A particularly suitable promoter can be, for example, a T. reesei cellobiohydrolase, endoglucanase, or beta-glucosidase promoter. For example, the promoter is a cellobiohydrolase I (cbh1) promoter. Non-limiting examples of promoters include a cbh1, cbh2, egl1, egl2, egl3, egl4, egl5, pki1, gpd1, xyn1, xyn2, or xyn3 promoter. Additional non-limiting examples of promoters include a T. reesei cbh1, cbh2, egl1, egl2, egl3, egl4, egl5, pki1, gpd1, xyn1, xyn2, or xyn3 promoter.

The nucleic acid sequence encoding a Mal3A polypeptide herein can be included in a vector. In some aspects, the vector contains the nucleic acid sequence encoding the Mal3A polypeptide under the control of an expression control sequence. In some aspects, the expression control sequence is a native expression control sequence. In some aspects, the expression control sequence is a non-native expression control sequence. In some aspects, the vector contains a selective marker or selectable marker. In some aspects, the nucleic acid sequence encoding the Mal3A polypeptide is integrated into a chromosome of a host cell without a selectable marker.

Suitable vectors are those which are compatible with the host cell employed. Suitable vectors can be derived, for example, from a bacterium, a virus (such as bacteriophage T7 or a M-13 derived phage), a cosmid, a yeast, or a plant. Suitable vectors can be maintained in low, medium, or high copy number in the host cell. Protocols for obtaining and using such vectors are known to those in the art (see, for example, Sambrook et al., Molecular Cloning: A Laboratory Manual, 2^{nd} ed., Cold Spring Harbor, 1989).

In some aspects, the expression vector also includes a termination sequence.

Termination control regions may also be derived from various genes native to the host cell. In some aspects, the termination sequence and the promoter sequence are derived from the same source.

An nucleic acid sequence encoding a Mal3A polypeptide can be incorporated into a vector, such as an expression vector, using standard techniques (Sambrook et al., Molecular Cloning: A Laboratory Manual, Cold Spring Harbor, 1982).

In some aspects, it may be desirable to over-express a Mal3A polypeptide and/or one or more of any other nucleic acid described in the present disclosure at levels far higher than currently found in naturally-occurring cells. In some embodiments, it may be desirable to under-express (e.g., mutate, inactivate, or delete) an endogenous beta-glucosidase and/or one or more of any other nucleic acid described in the present disclosure at levels far below that those currently found in naturally-occurring cells.

B. mal3a Polynucleotides

Another aspect of the compositions and methods described herein is a polynucleotide or a nucleic acid sequence that encodes a recombinant Mal3A polypeptide (including variants and fragments thereof) having beta-glucosidase activity. In some embodiments the polynucleotide is provided in the context of an expression vector for directing the expression of a Mal3A polypeptide in a heterologous organism, such as one identified herein. The polynucleotide that encodes a recombinant Mal3A polypeptide may be operably-linked to regulatory elements (e.g., a promoter, terminator, enhancer, and the like) to assist in expressing the encoded polypeptides.

An example of a polynucleotide sequence encoding a recombinant Mal3A polypeptide has the nucleotide sequence of SEQ ID NO: 1. Similar, including substantially identical, polynucleotides encoding recombinant Mal3A polypeptides and variants may occur in nature, e.g., in other strains or isolates of Melanocarpus albomyces, or Melanocarpus sp. In view of the degeneracy of the genetic code, it will be appreciated that polynucleotides having different nucleotide sequences may encode the same Mal3A polypeptides, variants, or fragments.

In some embodiments, polynucleotides encoding recombinant Mal3A polypeptides have a specified degree of amino acid sequence identity to the exemplified polynucleotide encoding a Mal3A polypeptide, e.g., at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% sequence identity to the amino acid sequence of SEQ ID NO: 2. Homology can be determined by amino acid sequence alignment, e.g., using a program such as BLAST, ALIGN, or CLUSTAL, as described herein.

In some embodiments, the polynucleotide that encodes a recombinant Mal3A polypeptide is fused in frame behind (i.e., downstream of) a coding sequence for a signal peptide for directing the extracellular secretion of a recombinant Mal3A polypeptide. As described herein, the term “heterologous”, when used to refer to a signal sequence used to express a polypeptide of interest, is meant that the signal sequence and the polypeptide of interest are from different organisms. Heterologous signal sequences include, for example, those from other fungal cellulase genes, such as, e.g., the signal sequence of T. reesei Bgl1 (SEQ ID NO:7) (amino acids 1 to 19 of SEQ ID NO:4). Expression vectors may be provided in a heterologous host cell suitable for expressing a recombinant Mal3A polypeptide, or suitable for propagating the expression vector prior to introducing it into a suitable host cell.

In some embodiments, polynucleotides encoding recombinant Mal3A polypeptides hybridize to the polynucleotide of SEQ ID NO: 1 (or to the complement thereof) under specified hybridization conditions. Examples of conditions are intermediate stringency, high stringency and extremely high stringency conditions, which are described herein.

Mal3A polynucleotides may be naturally occurring or synthetic (i.e., man-made), and may be codon-optimized for expression in a different host, mutated to introduce cloning sites, or otherwise altered to add functionality.

C. Vectors and Host Cells

In order to produce a disclosed recombinant Mal3A polypeptide, the DNA encoding the polypeptide can be chemically synthesized from published sequences or can be obtained directly from host cells harboring the gene (e.g., by cDNA library screening or PCR amplification). In some embodiments, the Mal3A polynucleotide is included in an expression cassette and/or cloned into a suitable expression vector by standard molecular cloning techniques. Such expression cassettes or vectors contain sequences that assist initiation and termination of transcription (e.g., promoters and terminators), and typically can also contain one or more selectable markers.

The expression cassette or vector is introduced into a suitable expression host cell, which then expresses the corresponding mal3a polynucleotide. Suitable expression hosts may be bacterial or fungal microbes. Bacterial expression host may be, for example, Escherichia (e.g., Escherichia coli), Pseudomonas (e.g., P. fluorescens or P. stutzerei), Proteus (e.g., Proteus mirabilis), Ralstonia (e.g., Ralstonia eutropha), Streptomyces, Staphylococcus (e.g., S. carnosus), Lactococcus (e.g., L. lactis), or Bacillus (e.g., Bacillus subtilis, Bacillus megaterium, Bacillus licheniformis, etc.). Fungal expression hosts may be, for example, yeasts, which can also serve as ethanologens. Yeast expression hosts may be, for example, Saccharomyces cerevisiae, Schizosaccharomyces pombe, Yarrowia lipolytica, Hansenula polymorpha, Kluyveromyces lactis or Pichia pastoris. Fungal expression hosts may also be, for example, filamentous fungal hosts including Aspergillus niger, Chrysosporium lucknowense, Myceliophthora thermophila, Aspergillus (e.g., A. oryzae, A. niger, A. nidulans, etc.) or T. reesei. Also suited are mammalian expression hosts such as mouse (e.g., N50), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing the Mal3A polypeptide.

Promoters and/or signal sequences associated with secreted proteins in a particular host of interest are candidates for use in the heterologous production and secretion of Mal3A polypeptides in that host or in other hosts. As an example, in filamentous fungal systems, the promoters that drive the genes for cellobiohydrolase I (cbh1), glucoamylase A (glaA), TAKA-amylase (amyA), xylanase (ex1A), the gpd-promoter cbh1, cbhll, endoglucanase genes eg1-eg5, Cel61B, Cel74A, gpd promoter, Pgk1, pki1, EF-1alpha, tef1, cDNA1 and hex1 are suitable and can be derived from a number of different organisms (e.g., A. niger, T. reesei, A. oryzae, A. awamori, A. nidulans).

In some embodiments, the Mal3A polynucleotide is recombinantly associated with a polynucleotide encoding a suitable homologous or heterologous signal sequence that leads to secretion of the recombinant Mal3A polypeptide into the extracellular (or periplasmic) space, thereby allowing direct detection of enzyme activity in the cell supernatant (or periplasmic space or lysate). Suitable signal sequences for Escherichia coli, other Gram negative bacteria and other organisms known in the art include those that drive expression of the HlyA, DsbA, Pbp, PhoA, PelB, OmpA, OmpT or M13 phage Gill genes. For Bacillus subtilis, Gram-positive organisms and other organisms known in the art, suitable signal sequences further include those that drive expression of the AprE, NprB, Mpr, AmyA, AmyE, Blac, SacB, and for S. cerevisiae or other yeast, including the killer toxin, Bar1, Suc2, Mating factor alpha, Inu1A or Ggplp signal sequence. Signal sequences can be cleaved by a number of signal peptidases, thus removing them from the rest of the expressed protein. Fungal expression signal sequences may be one that is selected from, for example, SEQ ID NOs: 6 to 34, 36 and 38.

In some embodiments, the recombinant Mal3A polypeptide is expressed alone or as a fusion with other peptides, tags or proteins located at the N- or C-terminus (e.g., 6×His, HA or FLAG tags). Suitable fusions include tags, peptides or proteins that facilitate affinity purification or detection (e.g., 6×His, HA, chitin binding protein, thioredoxin or FLAG tags), as well as those that facilitate expression, secretion or processing of the target beta-glucosidases Suitable processing sites include enterokinase, STE13, Kex2 or other protease cleavage sites for cleavage in vivo or in vitro.

Mal3A polynucleotides are introduced into expression host cells by a number of transformation methods including, but not limited to, electroporation, lipid-assisted transformation or transfection (“lipofection”), chemically mediated transfection (e.g. CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

D. Cell Culture Media

Generally, the microorganism is cultivated in a cell culture medium suitable for production of the Mal3A polypeptides described herein. The cultivation takes place in a suitable nutrient medium comprising carbon and nitrogen sources and inorganic salts, using procedures and variations known in the art. Suitable culture media, temperature ranges and other conditions for growth and cellulase production are known in the art. As a non-limiting example, a typical temperature range for the production of cellulases by T. reesei is 24° C. to 37° C., for example, between 25° C. and 30° C.

Materials and methods suitable for the maintenance and growth of fungal cultures are well known in the art. In some aspects, the cells are cultured in a culture medium under conditions permitting the expression of one or more beta-glucosidase polypeptides encoded by a nucleic acid inserted into the host cells. Standard cell culture conditions can be used to culture the cells. In some aspects, cells are grown and maintained at an appropriate temperature, gas mixture, and pH. In some aspects, cells are grown at in an appropriate cell medium.

### IV. Activities of Mal3A

The recombinant Mal3A polypeptides described herein have beta-glucosidase activity and/or a capacity to hydrolyze cellobiose and liberating D-glucose therefrom (cellobiase activity). As shown in the Examples below, the beta-glucosidase activity and/or the capacity to liberate D-glucose from cellobiose of Mal3A is improved (higher) than that observed from the benchmark high fidelity beta-glucosidase Bgl1 of T. reesei under the same conditions. With respect to cellobiase activity, Mal3A has at least 115%, 120%, 125%, or 127% of the activity observed with T. reesei Bgl1 (Example 3). In addition, the recombinant Mal3A polypeptide, as compared to the T. reesei Bgl1, is expected to have a high thermal activity profile and/or a high optimal temperature. Therefore, the Mal3A polypeptides described herein have improved functionality as compared to T. reesei Bgl1.

### V. Compositions Comprising a Recombinant Mal3A Beta-Glucosidase Polypeptide

The present disclosure provides engineered enzyme compositions (e.g., cellulase compositions) or fermentation broths enriched with a recombinant Mal3A polypeptides. In some aspects, the composition is a cellulase composition. The cellulase composition can be, e.g., a filamentous fungal cellulase composition, such as a Trichoderma cellulase composition. In some aspects, the composition is a cell comprising one or more nucleic acids encoding one or more cellulase polypeptides. In some aspects, the composition is a fermentation broth comprising cellulase activity, wherein the broth is capable of converting greater than about 50% by weight of the cellulose present in a biomass sample into sugars. The term “fermentation broth” and “whole broth” as used herein refers to an enzyme preparation produced by fermentation of an engineered microorganism that undergoes no or minimal recovery and/or purification subsequent to fermentation. The fermentation broth can be a fermentation broth of a filamentous fungus, for example, a Trichoderma, Humicola, Fusarium, Aspergillus, Neurospora, Penicillium, Cephalosporium, Achlya, Podospora, Endothia, Mucor, Cochliobolus, Pyricularia, Myceliophthora or Chrysosporium fermentation broth. In particular, the fermentation broth can be, for example, one of Trichoderma spp. such as a Trichoderma reesei, or Penicillium spp., such as a Penicillium funiculosum. The fermentation broth can also suitably be a cell-free fermentation broth. In one aspect, any of the cellulase, cell, or fermentation broth compositions of the present invention can further comprise one or more hemicellulases.

In some aspects, the whole broth composition is expressed in T. reesei or an engineered strain thereof. In some aspects the whole broth is expressed in an integrated strain of T. reesei wherein a number of cellulases including a Mal3A polypeptide has been integrated into the genome of the T. reesei host cell. In some aspects, one or more components of the polypeptides expressed in the integrated T. reesei strain have been deleted (see, e.g., T. reesei delete strains described in PCT application publication WO/2010/141779).

In some aspects, the whole broth composition is expressed in A. niger or an engineered strain thereof.

Alternatively, the recombinant Mal3A polypeptides can be expressed intracellularly. Optionally, after intracellular expression of the enzyme variants, or secretion into the periplasmic space using signal sequences such as those mentioned above, a permeabilisation or lysis step can be used to release the recombinant Mal3A polypeptide into the supernatant. The disruption of the membrane barrier is effected by the use of mechanical means such as ultrasonic waves, pressure treatment (French press), cavitation, or by the use of membrane-digesting enzymes such as lysozyme or enzyme mixtures. A variation of this embodiment includes the expression of a recombinant Mal3A polypeptide in an ethanologen microbe intracellularly. For example, a cellobiose transporter can be introduced through genetic engineering into the same ethanologen microbe such that cellobiose resulting from the hydrolysis of a lignocellulosic biomass can be transported into the ethanologen organism, and can therein be hydrolyzed and turned into D-glucose, which can in turn be metabolized by the ethanologen.

In some aspects, the polynucleotides encoding the recombinant Mal3A polypeptide are expressed using a suitable cell-free expression system. In cell-free systems, the polynucleotide of interest is typically transcribed with the assistance of a promoter, but ligation to form a circular expression vector is optional. In some embodiments, RNA is exogenously added or generated without transcription and translated in cell-free systems.

### VI. Uses of Mal3A Polypeptides to Hydrolyze a Lignocellulosic Biomass Substrate

In some aspects, provided herein are methods for converting lignocellulosic biomass to sugars, the method comprising contacting the biomass substrate with a composition disclosed herein comprising a Mal3A polypeptide in an amount effective to convert the biomass substrate to sugars (e.g., fermentable sugars). In some aspects, the method further comprises pre-treating the biomass prior to contacting it with the composition containing the Mal3A polypeptide, e.g., acid and/or base and/or mechanical (or other physical means) pre-treatment. In some aspects, an acid pre-treatment comprises contacting the biomass with phosphoric acid. In some aspects, the base comprises sodium hydroxide or ammonia. In some aspects, the mechanical means may include, for example, pulling, pressing, crushing, grinding, and other means of physically breaking down the lignocellulosic biomass into smaller physical forms. Other physical means may also include, for example, using steam or other pressurized fume or vapor to “loosen” the lignocellulosic biomass in order to increase accessibility by the enzymes to the cellulose and hemicellulose. In certain embodiments, the method of pretreatment may also involve enzymes that are capable of breaking down the lignin of the lignocellulosic biomass substrate, such that the accessibility of the enzymes of the biomass hydrolyzing enzyme composition to the cellulose and the hemicelluloses of the biomass is increased.

A. Biomass

The disclosure provides methods and processes for biomass saccharification, using the enzyme compositions of the disclosure, comprising a Mal3A polypeptide. The term “biomass,” as used herein, refers to any composition comprising cellulose and/or hemicellulose (optionally also lignin in lignocellulosic biomass materials). As used herein, biomass includes, without limitation, seeds, grains, tubers, plant waste (such as, for example, empty fruit bunches of the palm trees, or palm fibre wastes) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The disclosure therefore provides methods of saccharification comprising contacting a composition comprising a biomass material, for example, a material comprising xylan, hemicellulose, cellulose, and/or a fermentable sugar, with a Mal3A polypeptide of the disclosure, or a Mal3A polypeptide encoded by a nucleic acid or polynucleotide of the disclosure, or any one of the cellulase or non-naturally occurring hemicellulase compositions comprising a Mal3A polypeptide, or products of manufacture of the disclosure.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

For example, the process of converting a lignocellulosic biomass substrate to an ethanol can, in some embodiments, comprise two beta-glucosidase activities. For example, a first beta-glucosidase activity may be applied to the lignocellulosic biomass substrate during the saccharification or hydrolysis step, and a second beta-glucosidase activity can be applied as part of the ethanologen microbe in the fermentation step during which the monomeric or fermentable sugars that resulted from the saccharification or hydrolysis step are metabolized. The first and second beta-glucosidase activities may, in some embodiments, result from the presence of the same beta-glucosidase polypeptide. For example, the first beta-glucosidase activity in the saccharification may result from the presence of a Mal3A polypeptide of the invention, whereas the second beta-glucosidase activity in the fermentation stage may result from the expression of a different beta-glucosidase by the ethanologen microbe. In another example, the first and second beta-glucosidase activities may result from the presence of the same polypeptide in the saccharification or hydrolysis step and the fermentation step. For example, the same Mal3A polypeptide of the invention may, in some embodiments, provide the beta-glucosidase activities for both the hydrolysis or saccharification step and the fermentation step.

In certain other embodiments, the process of converting a lignocellulosic biomass substrate to an ethanol can, comprise two beta-glucosidase activities whereas the saccharification or hydrolysis step and the fermentation step occurs simultaneously, for example, in the same tank. Two or more beta-glucosidase polypeptides may contribute to the beta-glucosidase activities, one of which may be a Mal3A polypeptide of the invention.

In certain further embodiments, the process of converting a lignocellulosic biomass to an ethanol can comprise a single beta-glucosidase activity whereas either the saccharification or hydrolysis step or the fermentation step, but not both steps involves the participation of a beta-glucosidase. For example, a Mal3A polypeptide of the invention or a composition comprising the Mal3A polypeptide may be used in the saccharification step. In another example, the enzyme composition that is used to hydrolyze the lignocellulosic biomass substrate does not comprise a beta-glucosidase activity, whereas the ethanologen microbe expresses a beta-glucosidase polypeptide, for example, a Mal3A polypeptide of the invention.

B. Pretreatment

Prior to saccharification or enzymatic hydrolysis and/or fermentation of the fermentable sugars resulting from the saccharification, biomass (e.g., lignocellulosic material) is preferably subject to one or more pretreatment step(s) in order to render xylan, hemicellulose, cellulose and/or lignin material more accessible or susceptible to the enzymes in the enzymatic composition (for example, the enzymatic composition of the present invention comprising a Mal3A polypeptide) and thus more amenable to hydrolysis by the enzyme(s) and/or the enzyme compositions.

In some aspects, a suitable pretreatment method may involve subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506 and 6,423,145.

In some aspects, a suitable pretreatment method may involve subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

In further aspects, a suitable pretreatment method may involve processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841.

In yet further aspects, a suitable pretreatment method may involve pre-hydrolyzing biomass (e.g., lignocellulosic materials) in a pre-hydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. In a variation of this aspect, the pre-hydrolyzing can alternatively or further involves pre-hydrolysis using enzymes that are, for example, capable of breaking down the lignin of the lignocellulosic biomass material.

In yet further aspects, suitable pretreatments may involve the use of hydrogen peroxide H₂O₂. See Gould, 1984, Biotech, and Bioengr. 26:46-52.

In other aspects, pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34.

In some embodiments, pretreatment can comprise contacting a lignocellulose with a chemical (e.g., a base, such as sodium carbonate or potassium hydroxide) at a pH of about 9 to about 14 at moderate temperature, pressure, and pH. See PCT Publication WO2004/081185. Ammonia is used, for example, in a pretreatment method. Such a pretreatment method comprises subjecting a biomass material to low ammonia concentration under conditions of high solids. See, e.g., U.S. Patent Publication No. 20070031918 and PCT publication WO 06110901.

C. The Saccharification Process

In some aspects, provided herein is a saccharification process comprising treating biomass with an enzyme composition comprising a polypeptide, wherein the polypeptide has beta-glucosidase activity and wherein the process results in at least about 50 wt. % (e.g., at least about 55 wt. %, 60 wt. %, 65 wt. %, 70 wt. %, 75 wt. %, or 80 wt. %) conversion of biomass to fermentable sugars. In some aspects, the biomass comprises lignin. In some aspects the biomass comprises cellulose. In some aspects the biomass comprises hemicellulose. In some aspects, the biomass comprising cellulose further comprises one or more of xylan, galactan, or arabinan. In some aspects, the biomass may be, without limitation, seeds, grains, tubers, plant waste (e.g., empty fruit bunch from palm trees, or palm fibre waste) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like), potatoes, soybean (e.g., rapeseed), barley, rye, oats, wheat, beets, and sugar cane bagasse. In some aspects, the material comprising biomass is subject to one or more pretreatment methods/steps prior to treatment with the polypeptide. In some aspects, the saccharification or enzymatic hydrolysis further comprises treating the biomass with an enzyme composition comprising a Mal3A polypeptide of the invention. The enzyme composition may, for example, comprise one or more other cellulases, in addition to the Mal3A polypeptide. Alternatively, the enzyme composition may comprise one or more other hemicellulases. In certain embodiments, the enzyme composition comprises a Mal3A polypeptide of the invention, one or more other cellulases, one or more hemicellulases. In some embodiments, the enzyme composition is a whole broth composition.

In some aspects, provided is a saccharification process comprising treating a lignocellulosic biomass material with a composition comprising a polypeptide, wherein the polypeptide has at least about 75% (e.g., at least about 75%, 80%, 85%, 90%, 91%, 92%, 93%, 94%, 95%, 96%, 97%, 98%, 99%) sequence identity to SEQ ID NO:2, and wherein the process results in at least about 50% (e.g., at least about 55%, 60%, 65%, 70%, 75%, 80%, 85%, or 90%) by weight conversion of biomass to fermentable sugars. In some aspects, lignocellulosic biomass material has been subject to one or more pretreatment methods/steps as described herein.

Other aspects and embodiments of the present compositions and methods will be apparent from the foregoing description and following examples.

### EXAMPLES

The following examples are provided to demonstrate and illustrate certain embodiments and aspects of the present disclosure and should not be construed as limiting.

### Example 1

### Molecular Biology and Protein Production

A. Cloning & Expression of Benchmark T. reesei Bgl1 and Mal3A

a. Construction of the T. reesei Bgl1 Expression Vector

The N-terminal portion of the native T. reesei β-glucosidase gene bgl1 was codon optimized (DNA 2.0, Menlo Park, Calif.). This synthesized portion comprised the first 447 bases of the coding region of this enzyme. This fragment was then amplified by PCR using primers SK943 and SK941 (below). The remaining region of the native bgl1 gene was PCR amplified from a genomic DNA sample extracted from T. reesei strain RL-P37 (Sheir-Neiss, G et al. Appl. Microbiol. Biotechnol. 1984, 20:46-53), using the primers SK940 and SK942 (below). These two PCR fragments of the bgl1 gene were fused together in a fusion PCR reaction, using primers SK943 and SK942:

Forward Primer SK943:
(SEQ ID NO: 39)
5′-CACCATGAGATATAGAACAGCTGCCGCT-3′
Reverse Primer SK941:
(SEQ ID NO: 40)
5′-CGACCGCCCTGCGGAGTCTTGCCCAGTGGTCCCGCGACAG-3′
Forward Primer SK940:
(SEQ ID NO: 41)
5′-CTGTCGCGGGACCACTGGGCAAGACTCCGCAGGGCGGTCG-3′
Reverse Primer SK942:
(SEQ ID NO: 42)
5′-CCTACGCTACCGACAGAGTG-3′

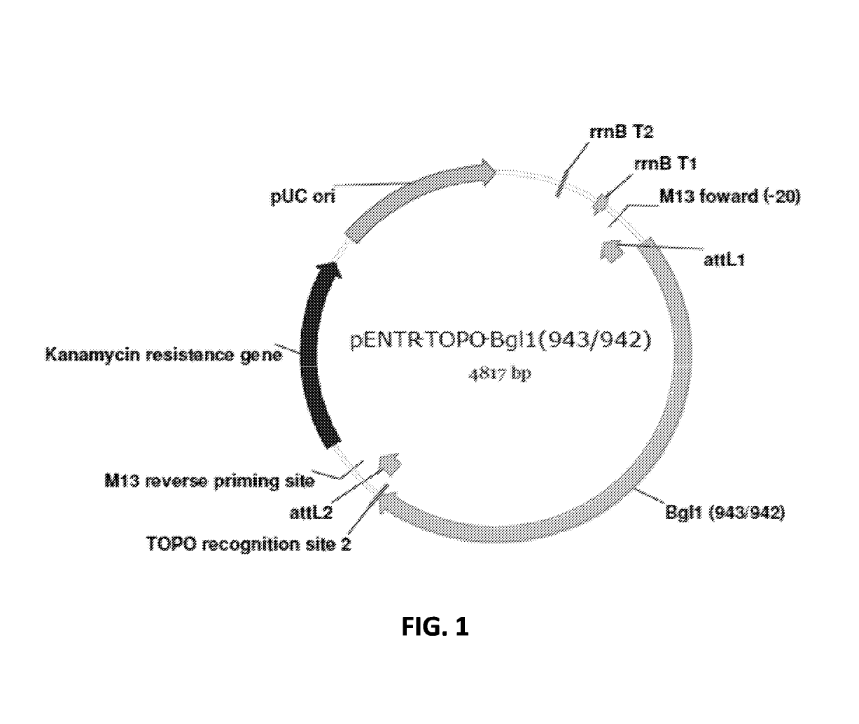

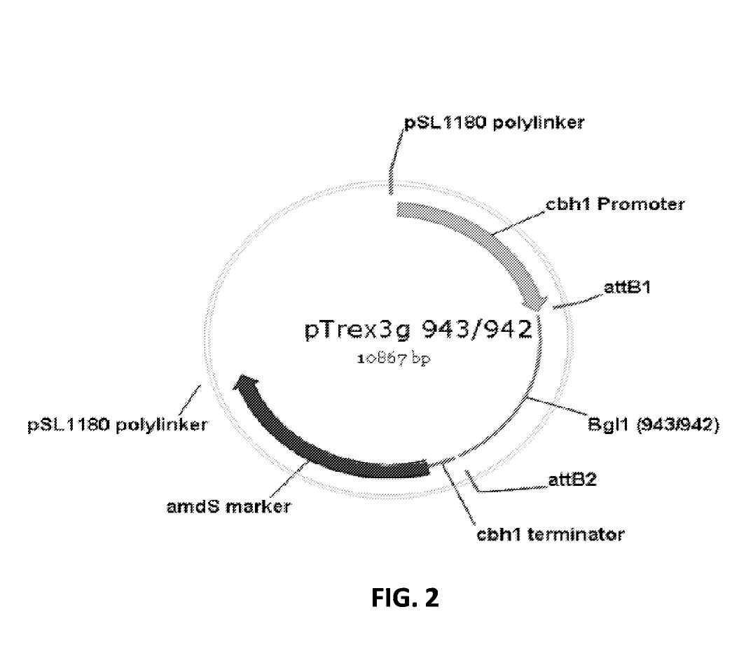

The resulting fusion PCR fragments were cloned into the Gateway® Entry vector pENTR™/D-TOPO® and transformed into E. coli One Shot® TOP10 Chemically Competent cells (Invitrogen) resulting in the intermediate vector, pENTR TOPO-Bgl1(943/942) (FIG. 1). The nucleotide sequence of the inserted DNA was determined. The pENTR-943/942 vector with the correct bgl1 sequence was recombined with pTrex3g using a LR Clonase® reaction (see, protocols outlined by Invitrogen). The LR clonase reaction mixture was transformed into E. coli One Shot®TOP10 Chemically Competent cells (Invitrogen), resulting in the expression vector, pTrex3g 943/942 (FIG. 2). The vector also contained the Aspergillus nidulans amdS gene, encoding acetamidase, as a selectable marker for transformation of T. reesei. The expression cassette was PCR amplified with primers SK745 and SK771 (below) to generate the product for transformation.

Forward Primer SK771:
(SEQ ID NO: 43)
5′-GTCTAGACTGGAAACGCAAC-3′
Reverse Primer SK745:
(SEQ ID NO: 44)
5′-GAGTTGTGAAGTCGGTAATCC-3′

b. Construction of the mal3A Expression Vector

The mal3A gene was PCR amplified from genomic DNA extracted from Melanocarpus albomyces strain CBS 638.94 using the JC_0138 and JC_0139 primers shown below:

Forward primer for Mal3A:
(SEQ ID NO: 45)
JC_0138-(5′-CAC CAT GAA GGC CGC TCT-3′)
Reverse primer for Mal3A:
(SEQ ID NO: 46)
JC_0139-(5′-CTA AGG CAA GGC AGC ACT CA-3′).

PCR conditions: (1) 94° C. 1 minute; (2) 94° C. 25 seconds; (3) 56° C. 25 seconds; (4) 72° C. 3 minutes; (5) Repeat steps 2-4, 24 times, −0.3° C. at step 3 per cycle; (6) Hold 4° C.

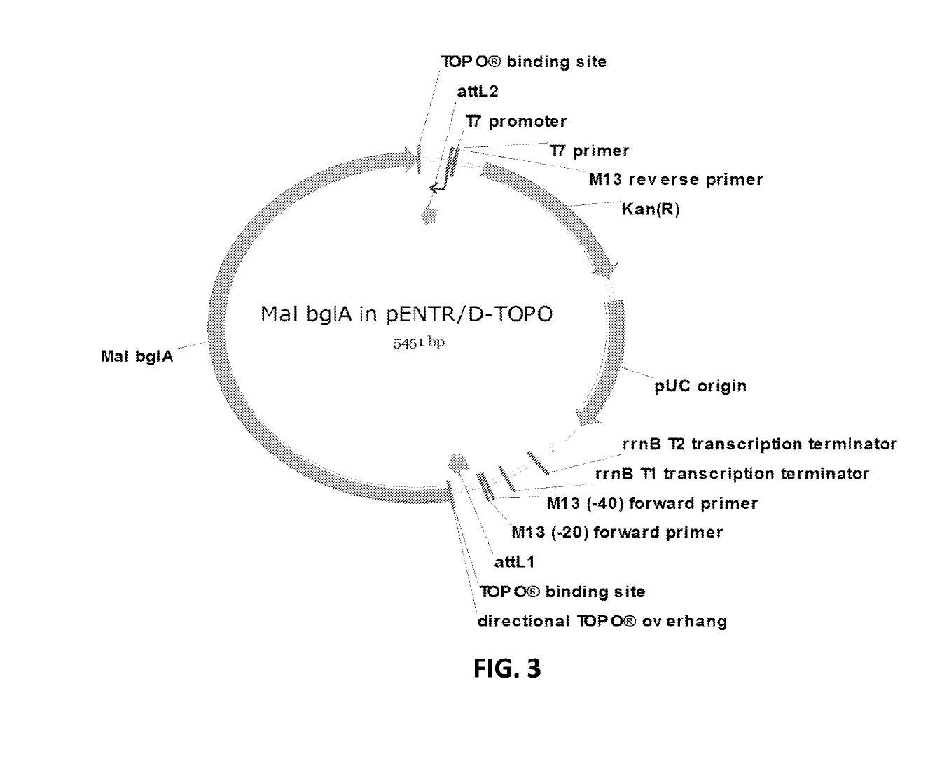

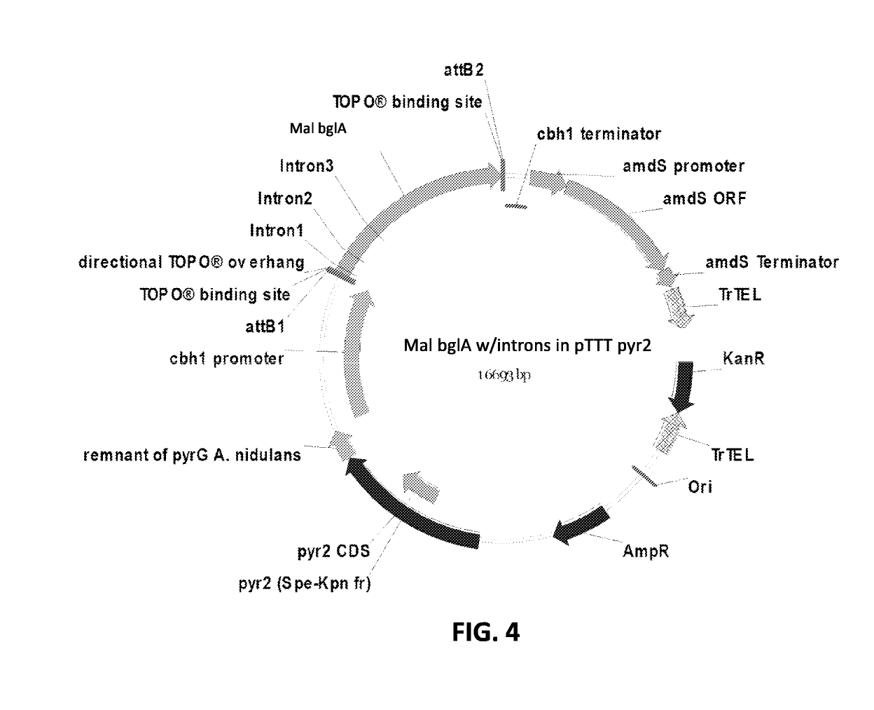

The resulting PCR fragment was cloned into the Gateway® Entry vector pENTR™/D-TOPO® and transformed into E. coli One Shot® TOP10 Chemically Competent cells (Invitrogen) resulting in the intermediate vector Mal_bglA in pENTR/D-TOPO (FIG. 3). The nucleotide sequence of the inserted DNA was determined and verified. The Mal_bglA in pEnter/D-TOPO with the correct mal3A sequence was recombined with the expression vector pTTT using a LR Clonase® reaction according to the manufacturer's instructions. The LR clonase reaction mixture was transformed into E. coli One Shot®TOP10 Chemically Competent cells (Invitrogen), resulting in the expression vector Mal_bglA in pTTT pyr2 (FIG. 4). In this expression vector, the mal3A gene is flanked with the cbh1 promoter and cbh1 terminator and also contains the Aspergillus nidulans amdS gene, encoding acetamidase, as a selectable marker for transformation of T. reesei. This plasmid was used for transformation into T. reesei.

c. Transformation of Mal3A into T. reesei

The plasmid Mal_bglA in pTTT pyr2 was transformed into a ten-gene deleted (cel7B, cel5A, cel6A, cel7A, cel3A, cel12A, cel45A, cel74A, man1, and cel61A) T. reesei host strain by the PEG-mediated protoplast method using amdS as the selectable marker (see PCT application publication WO/2010/141779 for a description of a quad-delete T. reesei strain in which genes encoding cellobiohydrolase I (cel7a), cellobiohydrolase II (cel6a), endoglucanase I (cel7b), and endoglucanase II (cel5a) have been inactivated as well as a description of methods for inactivating additional genes in T. reesei).

For protoplast preparation, spores were grown for 16-24 h at 24° C. in Trichoderma Minimal Medium MM, which contained 20 g/L glucose, 15 g/L KH₂PO₄, pH 4.5, 5 g/L (NH₄)₂SO₄, 0.6 g/L MgSO₄×7H₂O, 0.6 g/L CaCl₂×2H₂O, 1 mL of 1000× T. reesei Trace elements solution (which contained 5 g/L FeSO₄×7H₂O, 1.4 g/L ZnSO₄×7H₂O, 1.6 g/L MnSO₄×H₂O, 3.7 g/L CoCl₂×6H₂O) with shaking at 150 rpm. Germinating spores were harvested by centrifugation and treated with 50 mg/mL of Glucanex G200 (Novozymes AG) solution to lyse the fungal cell walls. Further preparation of the protoplasts was performed in accordance with a method described by Penttila et al. Gene 61 (1987) 155-164. The transformation mixtures, which contained about 1 μg of DNA and 1-5×10⁷ protoplasts in a total volume of 200 μL, were each treated with 2 mL of 25% PEG solution, diluted with 2 volumes of 1.2 M sorbitol/10 mM Tris, pH7.5, 10 mM CaCl₂, mixed with 3% selective top agarose MM containing 5 mM uridine and 20 mM acetamide. The resulting mixtures were poured onto 2% selective agarose plates containing uridine and acetamide. The transformation agar plate, containing acetamide, was incubated for 1 week at 32° C. The transformants were then pooled and plated again on acetamide-containing agar and incubated for 1 week at 28° C. in a light incubator before being inoculated into liquid media for protein production.

d. Construction of a Yeast Shuttle Vector pSC11

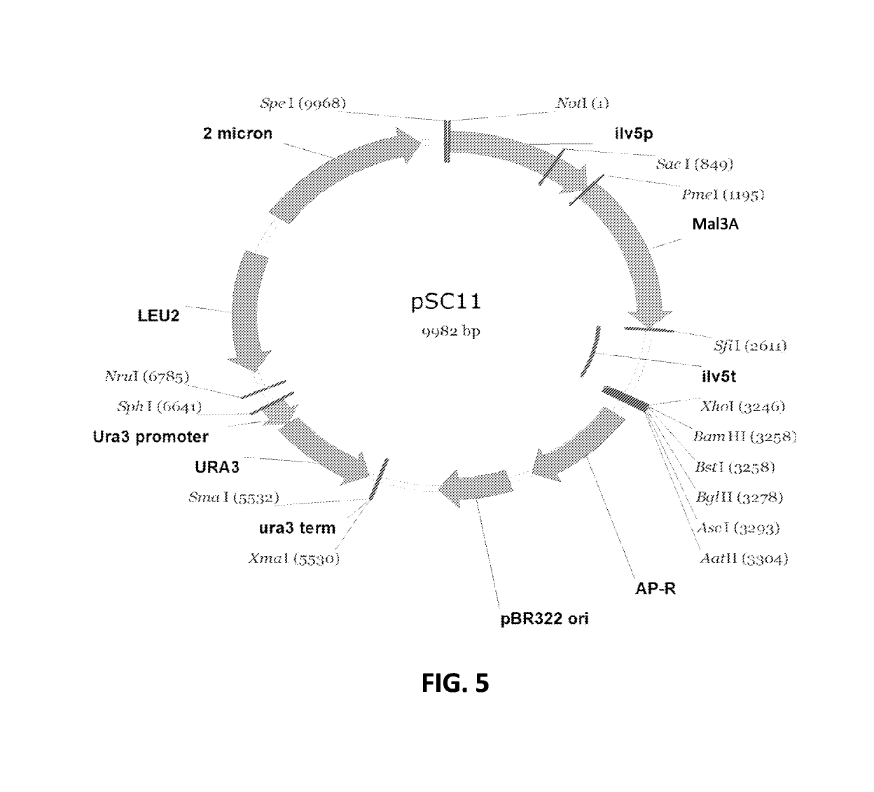

A yeast shuttle vector can be constructed in accordance with the vector map of FIG. 5. This vector can be used to express a Mal3A polypeptide in Saccharomyces cerevisiae intracellularly. A cellobiose transporter can be introduced into the Saccharomyces cerevisiae in the same shuttle vector or in a separate vector using known methods, such as, for example, those described by Ha et al., in PNAS, 108(2): 504-509 (2011).

Transformation of expression cassettes can be performed using the yeast EZ-Transformation kit. Transformants can be selected using YSC medium, which contains 20 g/L cellobiose. The successful introduction of the expression cassettes into yeast can be confirmed by colony PCR with specific primers.

Yeast strains can be cultivated in accordance with known methods and protocols. For example, they can be cultivated at 30° C. in a YP medium (10 g/L yeast extract, 20 g/L Bacto peptone) with 20 g/L glucose. To select transformants using an amino acid auxotrophic marker, yeast synthetic complete (YSC) medium may be used, which contains 6.7 g/L yeast nitrogen base plus 20 g/L glucose, 20 g/L agar, and CSM-Leu-Trp-Ura to supply nucleotides and amino acids.

e. Construction of a Zymomonas mobilis Integration Vector pZC11.

A Zymomonas mobilis integration vector pZC11 can be constructed in accordance with the vector map of FIG. 4. This vector can be used to express a Mal3A polypeptide in Zymomonas mobilis intracellularly. A cellobiose transporter can be introduced into the Zymomonas mobilis in the same integration vector or in a separate vector using known methods of introducing those transporters into a bacterial cell, such as, for example, those described by Sekar et al., Appl. Environ. Microbiol. (22 Dec. 2011).

Successful introduction of the integration vector as well as the cellobiose transporter gene can be confirmed using various known approaches, for example by PCR using confirmatory primers specifically designed for this purpose.

Zymomonas mobilis strains can be cultivated and fermented according to known methods, such as, for example, those described in U.S. Pat. No. 7,741,119.

f. Production and Purification of T. reesei Bgl1

T. reesei Bgl1 was over-expressed in, and purified from, the fermentation broth of a six-gene deleted (cel7B, cel5A, cel6A, cel7A, cel3A, cel12A) T. reesei host strain. A concentrated broth was loaded onto a G25 SEC column (GE Healthcase Bio-Sciences) and was buffer-exchanged against 50 mM sodium acetate, pH 5.0. The buffer exchanged Bgl1 was then loaded onto a 25 mL column packed with amino benzyl-S-glucopyranosyl sepharose affinity matrix. After extensive washing with 250 mM sodium chloride in 50 mM sodium acetate, pH 5.0, the bound fraction was eluted with 100 mM glucose in 50 mM sodium acetate and 250 mM sodium chloride, pH 5.0. The eluted fractions that tested positive for chloro-nitro-phenyl glucoside (CNPG) activity were pooled and concentrated. A single band corresponding to the MW of the T. reesei Bgl1 on SDS-PAGE and confirmed by mass spectrometry verified the purity of the eluted Bgl1. The final stock concentration was determined to be 2.2 mg/mL by absorbance at 280 nm.

g. Production and Purification of Mal3A

Mal3A was expressed by the ten-gene deleted T. reesei host strain noted in section c. above in 24-well slow-release microtiter plates (srMTPs) (see PCT Application No. PCT/US13/61051 entitled “Microtiter Plates for Controlled Release of Culture Components to Cell Cultures” filed on Sep. 20, 2013). Liquid minimal growth media contained 5 g/L (NH₄)₂SO₄; 4.5 g/L KH₂PO₄; 1.0 g/L MgSO₄.7H₂O; 33.0 g/L PIPPS; pH 5.5, with post-sterile addition of 1.6% glucose/sophorose mixture as the carbon source, 10 ml/L of 100 g/L of CaCl₂, 2.5 ml/L of T. reesei trace elements (400×): 175 g/L Citric acid anhydrous; 200 g/L FeSO₄.7H₂O; 16 g/L ZnSO₄.7H₂O; 3.2 g/L CuSO₄.5H₂O; 1.4 g/L MnSO₄.H₂O; 0.8 g/L H₃BO₃, in 24-well slow-release microtiter plate (80% [w/w] Polydimethylsiloxane [DOW Corning, Sylgard 184], 20% [w/w] lactose [Hilmar Ingredients, Hilmar 5020]). Secreted Mal3A protein expression was confirmed by SDS-PAGE (data not shown).

Mal3A was quantitated in the crude culture broth by UPLC analysis using a Waters ACQUITY UPLC C4BEH 300 Column as described below. Mal3A concentration as determined by UPLC analysis was 0.82 mg/mL. Mal3A was purified from the culture broth. One hundred (100) mLs of broth were desalted on a 500 ml G-25 column (GE Healthcare, PN17-0034-02), in 10 mM Tris (Tris[hydroxymethyl]aminomethane), pH 8.0. The desalted material was run on a 30 ml Resource Q column (SOURCE-15Q, GE Healthcare, PN17-0947-01). Buffer A was 10 mM Tris, pH 8.0; buffer B was 50 mM sodium acetate pH 5.0 plus 1 M NaCl. The gradient segments were 10-25%, 25-50%, 50-100%, 10 column volumes each. Five collected fractions were pooled and buffer exchanged into 50 mM MES (4-morpholineethanesulfonic acid) pH 6.0 plus 100 mM NaCl.

### Example 2

### Methods and Assays

A. Protein Concentration Measurement by UPLC

An Agilent HPLC 1290 Infinity system was used for protein quantitation with a Waters ACQUITY UPLC C4BEH 300 Column (1.7 μm, 1×50 mm). A six minute program with an initial gradient from 5% to 33% acetonitrile (Sigma-Aldrich) in 0.5 min, followed by a gradient from 33% to 48% in 4.5 min, and then a step gradient to 90% acetonitrile was used. A protein standard curve based on the purified T. reesei Bgl1 was used to quantify the Mal3A polypeptides.

B. Cellobiose Hydrolysis Assay

Cellobiose hydrolysis (cellobiase activity) was determined at 50° C. in either sodium acetate buffer (50 mM Na Acetate pH 5.0), or sodium citrate buffer with Tween 80 (50 mM Na Citrate pH 5.3 0.005% Tween 80) using the method described in Ghose, T. K. Pure & Applied Chemistry, 1987, 59 (2), 257-268. Briefly, 15 mM Cellobiose substrate (either in sodium acetate or sodium citrate buffer) was mixed at a 1:1 ratio with T. reesei Bgl1 or Mal3A in a 96-well microtiter plate (100 μL total volume, Costar #9017). The plate was covered and incubated in an Innova 44 incubator/shaker at 50° C. for 30 min. at 200 rpm. The reactions were quenched with 100 μl 100 mM Glycine, pH 10 buffer and mixed. Sugars were measured by HPLC (de-ashing column (Biorad 125-0118) and carbohydrate column (Aminex HPX-87P). The mobile phase was water, the flow rate was 0.6 ml/min, and the run time was 16 min/sample. Glucose standards from 0.1-1 mg/mL were used for converting peak area to concentration for all sugars.

Cellobiose units were derived as described in Ghose. Standard error for the cellobiase assay was determined to be 10%.

C. Hydrolysis of Dilute Ammonia Pretreated Corn Stover (daCS)

a. Production of daCS

Dilute Ammonia Pre-Treated Corn Stover (daCS) is prepared in accordance with a method described in published patent applications WO2004081185, US2007003198, and WO06110901. The prepared daCS is then diluted to 10% solids with 50 mM Sodium Citrate Buffer pH 5.3 (as described in the 50° C. assay below) or 50 mM Sodium Acetate Buffer pH 5.0 (as described in the 55° C. assay below) and stirred for several hours to overnight. The pH of the daCS slurry is adjusted to pH 5.3 if necessary using 1M Sodium Hydroxide. The carbohydrate composition is determined using the NREL Laboratory Analytical Procedure: Determination of Structural Carbohydrates and Lignin in Biomass (version 08-03-2012, see the nrel.gov website under “/biomass/analytical_procedures.html”). In some cases, unlike the NREL procedure, the biomass is dried at 50° C. and milled to <1 mm. The glucan and xylan content is determined and used to calculate percent conversion to total soluble sugars or to glucose or xylose monomer in the hydrolysis assays.

b. daCS at 50° C., pH 5.3

Saccharification performance is measured by creating dose curves for the Mal3A and a benchmark T. reesei Bgl1 (or other benchmark enzyme) in the presence of a fixed dose of a background whole cellulase composition (e.g., SPEZYME® CP whole cellulase; Danisco US Inc.). The whole cellulase background is used at 10 mg background/g glucan. Control assays are run using 10 mg and 20 mg of the background whole cellulase composition per gram of daCS with no added beta-glucosidase. The assay is performed in 96-well microtiter plates using 7% solids daCS in 50 mM Sodium Citrate Buffer, pH 5.3 and incubated at 50° C. with shaking (200 rpm) for two days. Each reaction has a final volume of 100 μL. Five replicates are run for each assay condition.

c. daCS at 55° C., pH 5.3

Saccharification performance is measured by creating dose curves for the Mal3A and the benchmark T. reesei Bgl1 in the presence of a fixed dose of a background whole cellulase composition (SPEZYME® CP whole cellulase; Danisco US Inc.). The whole cellulase background is used at 10 mg background/g glucan. Control assays include 10 mg and 20 mg of the background whole cellulase composition/gram daCS with no added beta-glucosidase. The assay is performed in 96-well microtiter plates using 7% solids daCS in 50 mM Sodium Acetate Buffer, pH 5.3 (as described above) and carried out at 55° C. with shaking (200 rpm) for two days. Each reaction has a final volume of 100 μL. Five replicates are run for each assay condition.

d. Measurement of Soluble Sugars from daCS Assays

The daCS microtiter plate assays described above (100 μL total volume in each well) is quenched by adding 100 μl of 100 mM Glycine buffer, pH 10. After mixing, the quenched reactions is transferred to a Millipore filter plate (Millipore, hydrophilic PVDF, 0.45 mm pores, cat. no. MAHVN4550) and placed on top of an HPLC plate (Agilent, cat. no. 5042-1385). The assembled plates are spun in a centrifuge for 5 min. according to manufacturer instructions. Soluble sugar levels (glucose, cellobiose and cellotriose) are measured in the filtered/spun samples by HPLC on an Agilent 100 series instrument using a de-ashing column (Biorad 125-0118) and a carbohydrate column (Aminex HPX-87P). Glucose standards (0.1-1 mg/mL) are used for converting peak area to concentration for all sugars.

D. Hydrolysis of Dilute Acid Pretreated Corn Stover (PCS)

a. Production of PCS

Dilute acid pretreated corn stover (PCS) was received from the National Renewable Energy Laboratory (NREL, Golden, Colo.; see the following reference for description of pre-treatment: Schell D J, Farmer J, Newman M, McMillan J D. Dilute-sulfuric acid pretreatment of corn stover in pilot scale reactor—Investigation of yields, kinetics and enzymatic digestibilities of solids. Appl Biochem Biotechnol. 2003; 105:69-85). The substrate was diluted by combining 20 g PCS (32.7% solids) with 40 ml of 50 mM Acetate buffer, pH 5.0. 60 μl of 5% sodium azide was added to discourage microbial growth. The slurry was mixed well, covered and allowed to gently stir at room temperature overnight. The pH of the substrate was then adjusted from 2.06 to 4.98 by adding 4 ml 1 M sodium hydroxide. Final solids were 10.2%, and this substrate was loaded into 96-well micro titer plates (Thermo Scientific #269787) for the saccharification assay.

b. PCS at 50° C., pH 5.0

T. reesei Bgl1 (TrBgl1) or Mal3A, both purified beta-glucosidases, were blended at 1% of the total protein with Spezyme® CP (Danisco US, Inc). The enzyme mixtures were dosed at 0, 2.5, 5, 10, and 20 mg protein/g glucan in PCS hydrolysis reactions in 50 mM Acetate buffer (pH 5.0) at 7% solids in 96-well microtiter plates. The PCS hydrolysis reactions were incubated at 50° C. for 2 days in an Innova 44 incubator/shaker (New Brunswick Scientific). Each dose was performed in quadruplicate (i.e., 4 reactions at each of 0, 2.5, 5, 10 and 20 mg protein/g glucan).

c. Measurement of Soluble Sugars from PCS Assay

Measurement of soluble sugars was performed as described above in Example 2-C-d. The mobile phase was water, with a flow rate of 0.6 ml/min, and a run time of 20 min/sample. Glucose standards (0.1-1 mg/ml) were used for converting peak area to concentration.

### Example 3

### Improved Cellobiose Hydrolysis Performance of Mal3A Over the Benchmark T. reesei Bgl1

The concentration of Mal3A present in the crude broth produced in srMTP was measured by UPLC (described herein) and determined to be 0.82 g/L. Purified T. reesei Bgl1 was used from a stock of 2.2 mg/mL (A280 measurement). The cellobiose hydrolysis activity of each enzyme was measured as described above in Example 2-B and is presented in Table 3-1 as activity relative to purified T. reesei Bgl1.

TABLE 3-1

| | Sodium Acetate | Sodium Citrate, Tween 80 |
|---|---|---|
| T. reesei Bgl1 | 1.0 | 1.0 |
| Mal3A (crude) | 1.19 | 1.27 |
| Mal3A (purified) | 1.22 | (Not done) |

As shown above, Mal3A demonstrated improved cellobiase activity as compared to T. reesei Bgl1 (19% improvement in activity as compared to the T. reesei Bgl1 standard in Sodium Acetate buffer; 27% improvement in activity as compared to the T. reesei Bgl1 standard in Sodium Citrate buffer plus Tween 80).

### Example 4

### Hydrolysis Performance of Mal3A Polypeptides on daCS at 50° C. as Compared to Benchmark T. reesei Bgl1

Dose curves of daCS hydrolysis activity at 50° C. are determined for T. reesei Bgl1 and Mal3A as described above in Example 2 C-b (i.e., in the presence of a fixed dose of background whole cellulase composition; SPEZYME® CP whole cellulase, Danisco US Inc.). T. reesei Bgl1 is tested at 0.1 mg/g glucan, 0.25 mg/g glucan, 0.5 mg/g glucan, 1.0 mg/g glucan, 2.5 mg/g glucan, and 7.5 mg/g glucan; Mal3A is tested at 0.19 mg/g glucan, 0.48 mg/g glucan, 0.95 mg/g glucan, 1.91 mg/g glucan, 3.81 mg/g glucan, 5.72 mg/g glucan, 7.63 mg/g glucan, and 9.53 mg/g glucan. Soluble sugar levels (glucose, cellobiose and cellotriose) are measured by HPLC as described above in Example 2 C-d.

The % glucan conversion for the above dose response curves is determined using the following formula:

$$\frac{\begin{matrix}( mg glucose released + mg cellobiose released + \\ mg cellotriose released )\end{matrix}}{mg cellulose in the starting daCS substrate}$$

It is expected that Mal3A outperforms T. reesei Bgl1 for glucan conversion of daCS at 50° C.

### Example 5

### Hydrolysis Performance of Mal3A Polypeptides on daCS at 55° C. as Compared to Benchmark T. reesei Bgl1

Dose curves of daCS hydrolysis activity at 55° C. is determined for T. reesei Bgl1 and Mal3A as described above in Example 2 C-c (i.e., in the presence of a fixed dose of background whole cellulase composition; SPEZYME® CP whole cellulase, Danisco US Inc.). T. reesei Bgl1 is tested at 0.1 mg/g glucan, 0.25 mg/g glucan, 0.5 mg/g glucan, 1.0 mg/g glucan, 2.5 mg/g glucan, 5.0 mg/g glucan, 7.5 mg/g glucan, and 10.0 mg/g glucan; Mal3A is tested at 0.1 mg/g glucan, 0.25 mg/g glucan, 0.5 mg/g glucan, 1.0 mg/g glucan, 2.5 mg/g glucan, 5.0 mg/g glucan, 7.5 mg/g glucan, and 9.5 mg/g glucan. Soluble sugar levels (glucose, cellobiose, cellotriose, xylose, and xylobiose) are measured by HPLC as described above in Example 2 C-d.

The % glucan conversion for the above dose response curves is determined using the following formula:

$$\frac{\begin{matrix}( mg glucose released + mg cellobiose released + \\ mg cellotriose released )\end{matrix}}{mg cellulose in the starting daCS substrate}$$

It is expected that Mal3A outperforms T. reesei Bgl1 for glucan conversion of daCS at 55° C.

### Example 6

### Hydrolysis of Mal3A Polypeptides on Acid Pre-Treated Corn Stover (PCS) at 50° C. as Compared to Benchmark T. reesei Bgl1

Dose curves of PCS hydrolysis activity at 50° C. were performed for T. reesei Bgl1 blended at 1% of the total protein with Spezyme® CP (Danisco US, Inc) and compared to the same dose curve for Mal3A blended at 1% of the total protein with Spezyme® CP (as described above in Example 2 D). These 1% blends were dosed at 0, 2.5, 5.0, 10.0, and 20.0 mg/g glucan and run in quadruplicate. Soluble sugar levels were measured by HPLC as described above in Example 2 D-c.

As shown in Table 6-1 below, Mal3A β-glucosidase exceeded Trichoderma reesei Bgl1 (TrBgl1) performance under these conditions. Specifically, 14 mg/g glucan of Mal3A was required to reach 60% conversion of the PCS substrate to soluble sugars while it took 15 mg/g glucan of TrBgl1 to reach this conversion percentage. In relative terms, it takes 7% less of a Spezyme CP+1% Mal3A blend than a Spezyme CP+1% TrBgl1 blend to achieve equivalent conversion.

TABLE 6-1

| | Dose to reach 60% conversion | Relative |
|---|---|---|
| Enzyme Blend | (mg protein/g glucan) | to TrBgl1 |
| Spezyme CP + 1% TrBgl1 | 15.0 | 1.00 |
| Spezyme CP + 1% Mal3A | 14.0 | 0.93 |
| Spezyme CP | 18.2 | 1.22 |

Although the foregoing compositions and methods have been described in some detail by way of illustration and example for purposes of clarity of understanding, it is readily apparent to those of ordinary skill in the art in light of the teachings herein that certain changes and modifications may be made thereto without departing from the spirit or scope of the appended claims.

Accordingly, the preceding merely illustrates the principles of the present compositions and methods. It will be appreciated that those skilled in the art will be able to devise various arrangements which, although not explicitly described or shown herein, embody the principles of the present compositions and methods and are included within its spirit and scope. Furthermore, all examples and conditional language recited herein are principally intended to aid the reader in understanding the principles of the present compositions and methods and the concepts contributed by the inventors to furthering the art, and are to be construed as being without limitation to such specifically recited examples and conditions. Moreover, all statements herein reciting principles, aspects, and embodiments of the present compositions and methods as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present compositions and methods, therefore, is not intended to be limited to the exemplary embodiments shown and described herein.

### Sequences

SEQ ID NO: 1: Genomic DNA sequence encoding Mal3A
atgaaggccgctcttgcggttgcctcgctgctcagcggcagtcttgctgccgcgggcacgctccatccac
gacacaaggtacggcaagctcgccgttccctggctggtgatggttcgggagtgagagtcccgtttgctga
cagtcaataaaatcacccatcagctcgcaaagagggccctcgcaacgtcggatcccttttacccctcgcc
atggatgaatcccgaggcagatggctgggcggaagcgtacgcccaggcgagggagttcgtctcgcagatg
acgctgctggagaaggtcaacctgaccaccggcaccgggtaagtgtcgcgggtggccgcccatacccgtg
gcgcctttcttctccatgcacgatgcgccgtgattctgacgattcgcagctgggcgtccgagcagtgcgt
gggcaacacaggctcaattcctcgcctcggtctccgcagcttgtgcttgcacgacgctccgcttggcatc
cgcgggtcggactacaactcggccttcccctcgggacagaccgtcgccgccacctttgaccgcactctga
tgtacaggcgcggctacgccatgggcctcgaggcgaagggcaagggcatcaacgtcctgctcgggccgtc
cgctggccccatcggccgcatgcctgccggcggccggaactgggaaggcttctcgccggatcccgtgctc
tcgggcattggcatggccgagtcggtcaagggcatccaggacgccggcgtgatcgcctgcgccaagcact
tcatcggcaacgagcaaggtaagtcgggtcgacacggccgcgggatatggggtggtggtggtggtggtgg
tgatggagagcccgccagctgacatggggatcagagcacttcagacaggtgggagaggccatcgggtacg
gcttcgacatcaccgagacgctgtcttccaacatcgacgacaggacgatgcacgagctctacctctggcc
cttcgcggatgctgtccgcgcgggcgtcggctccatcatgtgctcgtaccagcaggtcaacaactcgtat
gcctgccagaactccaaggtcctgaacgacctgctcaagaacgagctcggattccagggcttcgtcctga
gcgactggcaagcgcagcacacgggcgcggccagcgccgtcgccggtctcgacatgaccatgccgggaga
caccgagttcaacacgggcctcagctactggggcaccaacctcacgctcgccgtgctgaacggcaccgtc
ccggcctaccggatcgatgacatggccatgcgcatcatggccgccttcttcaaggtcagcaagagcatcg
acctggaccccatcaacttctccttctggacgctggacacgtacggcccgatccactgggccgcgaacga
gggccaccagcagatcaaccaccacgtcgacgtccggcagcccgaccacgcacacctcatccgcgagatc
ggcgccaagggcacggtgctgctgaagaacacggggtctctgcccctcgacaagcccaagttcctggccg
tcatcggcgaggacgccggcccgaaccccaggggccccaactcctgcgccgaccgcggctgcaacgaggg
cacgctcgccatgggctggggctcgggcacggccaacttcccgtacctggtgacgccggatgcggcgctg
caggcccaggccatccaggacggctcgcgatacgagagcatcctgtccaactacgcgctcgatgagacga
gggccctggtgtcgcaggaggatgccaccgcgatcgtcttcgtcaatgccaactcgggcgagggctacat
caacgtggacggcaacatgggcgaccgcaagaacctgacgctctggcggggcggcgacgacctggtcaag
aacgtgtcgagctggtgctccaacaccatcgtcgtcatccactccaccggccccgtcctcctgacagact
ggtacgacagccccaacatcacggcgatcctgtgggccggcctccccggccaggagtcgggcaactcgat
cgtcgatgtcctgtacggcaaggtcaacccggccggccgcacgcccttcacgtggggagcgacccgggag
ggctacggcgccgacgttctctacgagccgaacaacggcaacggcgcgccgcagcaggacttcaccgagg
gcgtcttcatcgactaccgctacttcgacaaggccaacacgtcggtcatctacgagttcggccacggcct
cagctacacgacgttcgagtacagcaacatccgggtggagaagcacaacgccggcccgtaccggccgacg
gagggcatgacggcgcccgcgccgacgtttggcaacttctcgaccgacctcgaggactacctgttcccgg
aggacgagttcccctacgtctaccagtacatctacccgtacctcaacacgacggaccccgagaaggcgtc
ggccgatccgcactacggccaggcggccgacgagttcctgccgccgcgcgccaccgacgactcggcgcag
ccgctcctgcgcgcgtcggacaggcacgcgcccggcggcaaccgcggcctgtacgacgtgctgtacaccg
tcacggccgacatcaccaacacgggccccatcgtcggggaggaggtgccgcagctctacgtctcgctcgg
cgggcccgacaaccccaaggtcgtcctccgcgactttgcccgcctgcgcatcgaccccggccagacggtc
cggttccgcggcaccctcacgaggagggacctgagcaactgggaccccgtcgtccaggactgggtcgtcg
gccgccacaacaagaccgtctatgtcggccggagcagccgcaagctggatctgagtgctgccttgccttg
a
SEQ ID NO: 2: Polypeptide sequence of Ma13A (underlined residues are
the predicted signal sequence)
MKAALAVASLLSGSLAAAGTLHPRHKLAKRALATSDPFYPSPWMNPEADGWAEAYAQAREFVSQMTLLEK
VNLTTGTGWASEQCVGNTGSIPRLGLRSLCLHDAPLGIRGSDYNSAFPSGQTVAATFDRTLMYRRGYAMG
LEAKGKGINVLLGPSAGPIGRMPAGGRNWEGFSPDPVLSGIGMAESVKGIQDAGVIACAKHFIGNEQEHF
RQVGEAIGYGFDITETLSSNIDDRTMHELYLWPFADAVRAGVGSIMCSYQQVNNSYACQNSKVLNDLLKN
ELGFQGFVLSDWQAQHTGAASAVAGLDMTMPGDTEFNTGLSYWGTNLTLAVLNGTVPAYRIDDMAMRIMA
AFFKVSKSIDLDPINFSFWTLDTYGPIHWAANEGHQQINHHVDVRQPDHAHLIREIGAKGTVLLKNTGSL
PLDKPKFLAVIGEDAGPNPRGPNSCADRGCNEGTLAMGWGSGTANFPYLVTPDAALQAQAIQDGSRYESI
LSNYALDETRALVSQEDATAIVFVNANSGEGYINVDGNMGDRKNLTLWRGGDDLVKNVSSWCSNTIVVIH
STGPVLLTDWYDSPNITAILWAGLPGQESGNSIVDVLYGKVNPAGRTPFTWGATREGYGADVLYEPNNGN
GAPQQDFTEGVFIDYRYFDKANTSVIYEFGHGLSYTTFEYSNIRVEKHNAGPYRPTEGMTAPAPTFGNFS
TDLEDYLFPEDEFPYVYQYIYPYLNTTDPEKASADPHYGQAADEFLPPRATDDSAQPLLRASDRHAPGGN
RGLYDVLYTVTADITNTGPIVGEEVPQLYVSLGGPDNPKVVLRDFARLRIDPGQTVRFRGTLTRRDLSNW
DPVVQDWVVGRHNKTVYVGRSSRKLDLSAALP
SEQ ID NO: 3: Mature Mal3A polypeptide sequence:
AAGTLHPRHKLAKRALATSDPFYPSPWMNPEADGWAEAYAQAREFVSQMTLLEKVNLTTGTGWASEQCVG
NTGSIPRLGLRSLCLHDAPLGIRGSDYNSAFPSGQTVAATFDRTLMYRRGYAMGLEAKGKGINVLLGPSA
GPIGRMPAGGRNWEGFSPDPVLSGIGMAESVKGIQDAGVIACAKHFIGNEQEHFRQVGEAIGYGFDITET
LSSNIDDRTMHELYLWPFADAVRAGVGSIMCSYQQVNNSYACQNSKVLNDLLKNELGFQGFVLSDWQAQH
TGAASAVAGLDMTMPGDTEFNTGLSYWGTNLTLAVLNGTVPAYRIDDMAMRIMAAFFKVSKSIDLDPINF
SFWTLDTYGPIHWAANEGHQQINHHVDVRQPDHAHLIREIGAKGTVLLKNTGSLPLDKPKFLAVIGEDAG
PNPRGPNSCADRGCNEGTLAMGWGSGTANFPYLVTPDAALQAQAIQDGSRYESILSNYALDETRALVSQE
DATAIVFVNANSGEGYINVDGNMGDRKNLTLWRGGDDLVKNVSSWCSNTIVVIHSTGPVLLTDWYDSPNI
TAILWAGLPGQESGNSIVDVLYGKVNPAGRTPFTWGATREGYGADVLYEPNNGNGAPQQDFTEGVFIDYR
YFDKANTSVIYEFGHGLSYTTFEYSNIRVEKHNAGPYRPTEGMTAPAPTFGNFSTDLEDYLFPEDEFPYV
YQYIYPYLNTTDPEKASADPHYGQAADEFLPPRATDDSAQPLLRASDRHAPGGNRGLYDVLYTVTADITN
TGPIVGEEVPQLYVSLGGPDNPKVVLRDFARLRIDPGQTVRFRGTLTRRDLSNWDPVVQDWVVGRHNKTV
YVGRSSRKLDLSAALP
SEQ ID NO: 4: T.reesei Bgl1 polypeptide sequence (underlined residues
are predicted signal sequence residues)
MRYRTAAALALATGPFARADSHSTSGASAEAVVPPAGTPWGTAYDKAKAALAKLNLQDKVGIVSGVGWNG
GPCVGNTSPASKISYPSLCLQDGPLGVRYSTGSTAFTPGVQAASTWDVNLIRERGQFIGEEVKASGIHVI
LGPVAGPLGKTPQGGRNWEGFGVDPYLTGIAMGQTINGIQSVGVQATAKHYILNEQELNRETISSNPDDR
TLHELYTWPFADAVQANVASVMCSYNKVNTTWACEDQYTLQTVLKDQLGFPGYVMTDWNAQHTTVQSANS
GLDMSMPGTDFNGNNRLWGPALTNAVNSNQVPTSRVDDMVTRILAAWYLTGQDQAGYPSFNISRNVQGNH
KTNVRAIARDGIVLLKNDANILPLKKPASIAVVGSAAIIGNHARNSPSCNDKGCDDGALGMGWGSGAVNY
PYFVAPYDAINTRASSQGTQVTLSNTDNTSSGASAARGKDVAIVFITADSGEGYITVEGNAGDRNNLDPW
HNGNALVQAVAGANSNVIVVVHSVGAIILEQILALPQVKAVVWAGLPSQESGNALVDVLWGDVSPSGKLV
YTIAKSPNDYNTRIVSGGSDSFSEGLFIDYKHFDDANITPRYEFGYGLSYTKFNYSRLSVLSTAKSGPAT
GAVVPGGPSDLFQNVATVTVDIANSGQVTGAEVAQLYITYPSSAPRTPPKQLRGFAKLNLTPGQSGTATF
NIRRRDLSYWDTASQKWVVPSGSFGISVGASSRDIRLTSTLSVA
SEQ ID NO: 5: Mature T.reesei Bgl1 polypeptide sequence
DSHSTSGASAEAVVPPAGTPWGTAYDKAKAALAKLNLQDKVGIVSGVGWNGGPCVGNTSPASKISYPSLC
LQDGPLGVRYSTGSTAFTPGVQAASTWDVNLIRERGQFIGEEVKASGIHVILGPVAGPLGKTPQGGRNWE
GFGVDPYLTGIAMGQTINGIQSVGVQATAKHYILNEQELNRETISSNPDDRTLHELYTWPFADAVQANVA
SVMCSYNKVNTTWACEDQYTLQTVLKDQLGFPGYVMTDWNAQHTTVQSANSGLDMSMPGTDFNGNNRLWG
PALTNAVNSNQVPTSRVDDMVTRILAAWYLTGQDQAGYPSFNISRNVQGNHKTNVRAIARDGIVLLKNDA
NILPLKKPASIAVVGSAAIIGNHARNSPSCNDKGCDDGALGMGWGSGAVNYPYFVAPYDAINTRASSQGT
QVTLSNTDNTSSGASAARGKDVAIVFITADSGEGYITVEGNAGDRNNLDPWHNGNALVQAVAGANSNVIV
VVHSVGAIILEQILALPQVKAVVWAGLPSQESGNALVDVLWGDVSPSGKLVYTIAKSPNDYNTRIVSGGS
DSFSEGLFIDYKHFDDANITPRYEFGYGLSYTKFNYSRLSVLSTAKSGPATGAVVPGGPSDLFQNVATVT
VDIANSGQVTGAEVAQLYITYPSSAPRTPPKQLRGFAKLNLTPGQSGTATFNIRRRDLSYWDTASQKWVV
PSGSFGISVGASSRDIRLTSTLSVA
SIGNAL PEPTIDE SEQUENCES (SEQ ID NOs: 6-34, 36 and 38 are amino acid
sequences; SEQ ID NO: 35 is polynucleotide sequence encoding SEQ ID NO:
36; SEQ ID NO: 37 is polynucleotide sequence encoding SEQ ID NO: 38)
SEQ ID NO: 6: Mal3A signal peptide sequence
MKAALAVASLLSGSLA
SEQ ID NO: 7: T. reesei Bgl1 signal peptide sequence
MRYRTAAALALATGPFARA
SEQ ID NO: 8:
MVSFTSLLAASPPSRASCRPAAEVESVAVEKR
SEQ ID NO: 9:
MKANVILCLLAPLVAA
SEQ ID NO: 10:
MIVGILTTLATLATLAAS
SEQ ID NO: 11:
MYRKLAVISAFLATARA
SEQ ID NO: 12:
MLLNLQVAASALSLSLLGGLAEA
SEQ ID NO: 13:
MKLNWVAAALSIGAAGTDS
SEQ ID NO: 14:
MASIRSVLVSGLLAAGVNA
SEQ ID NO: 15:
MWLTSPLLFASTLLGLTGVALA
SEQ ID NO: 16:
MRFSWLLCPLLAMGSA
SEQ ID NO: 17:
MRLLSFPSHLLVAFLTLKEASS
SEQ ID NO: 18:
MQLKFLSSALLLSLTGNCAA
SEQ ID NO: 19:
MKVYWLVAWATSLTPALA
SEQ ID NO: 20:
MVRFSSILAAAACFVAVES
SEQ ID NO: 21:
MIHLKPALAALLALSTQCVA
SEQ ID NO: 22:
MALQTFFLLAAAMLANA
SEQ ID NO: 23:
MKLNKPFLAIYLAFNLAEA
SEQ ID NO: 24:
MAPLSLRALSLLALTGAAAA
SEQ ID NO: 25:
MVRPTILLTSLLLAPFAAA
SEQ ID NO: 26:
MHMHSLVAALAAGTLPLLASA
SEQ ID NO: 27:
MVHLSSLAAALAALPLVYG
SEQ ID NO: 28:
MRFSLAATTLLAGLATA
SEQ ID NO: 29:
MVVLSKLVSSILFASLVSA
SEQ ID NO: 30:
MVQIKAAALAMLFASHVLS
SEQ ID NO: 31:
MKASSVLLGLAPLAALA
SEQ ID NO: 32:
MRFPSIFTAVLFAASSALA
SEQ ID NO: 33:
MRFPSIFTAVLFAASSALAAPVNTTTEDETAQIPAEAVIGYLDLEGDFDVAVLPFSNSTNNGLLFINTTI
ASIAAKEEGVSLDKR
SEQ ID NO: 34:
MLLQAFLFLLAGFAAKISAR
SEQ ID NO: 35:
ATGATAAAAGTCCCGCGGTTCATCTGTATGATCGCGCTTACATCCAGCGTTCTGGCAAGCGGCCTTTCTC
AAAGCGTTTCAGCTCAT
SEQ ID NO: 36 (AMINO ACID SESQUENCE ENCODED BY 41)
MIKVPRFICMIALTSSVLASGLSQSVSAH
SEQ ID NO: 37:
ATGAAAAGAAAGCTTGGTCGTCGCCAGTTATTAACTGGCTTTGTTGCCCTTGGCGGTATGGCGATTACAG
CTGGTAAGGCGCAGGCTTCT
SEQ ID NO: 38 (AMINO ACID SEQUENCE ENCODED BY 43)
MKRKLGRRQLLTGFVALGGMAITAGKAQAS
PRIMER SEQUENCES
SEQ ID NO: 39:
CACCATGAGATATAGAACAGCTGCCGCT
SEQ ID NO: 40
CGACCGCCCTGCGGAGTCTTGCCCAGTGGTCCCGCGACAG
SEQ ID NO: 41
CTGTCGCGGGACCACTGGGCAAGACTCCGCAGGGCGGTCG
SEQ ID NO: 42
CCTACGCTACCGACAGAGTG
SEQ ID NO: 43
GTCTAGACTGGAAACGCAAC
SEQ ID NO: 44
GAGTTGTGAAGTCGGTAATCC
SEQ ID NO: 45
CACCATGAAGGCCGCTCT
SEQ ID NO: 46
CTAAGGCAAGGCAGCACTCA
SEQ ID NO: 47
TGAGAGTCCCGTTTGCTGAC
SEQ ID NO: 48
TCGGTCAAGGGCATCCAGG
SEQ ID NO: 49
TTCAAGGTCAGCAAGAGCAT
SEQ ID NO: 50
CTGACAGACTGGTACGACAG
SEQ ID NO: 51
TACCAGTACATCTACCCGTA

1. A composition comprising
a) a recombinant polypeptide comprising an amino acid sequence that is at least 80% identical to the amino acid sequence of SEQ ID NO:2 or SEQ ID NO:3, wherein the polypeptide has beta-glucosidase activity; and
b) one or more cellulases or hemicellulases wherein the one or more cellullases or hemicellulases are not derived from a Melanocarpus albomyces strain.

2. The composition of claim 1, wherein the polypeptide has improved beta-glucosidase activity as compared to Trichoderma reesei Bgl1 when the recombinant polypeptide and the Trichoderma reesei Bgl1 are used to hydrolyze lignocellulosic biomass substrates.

3. The composition of claim 1, wherein the improved beta-glucosidase activity is an increased cellobiase activity.

4. The composition of claim 1, wherein the polypeptide comprises an amino acid sequence that is at least 80% identical to the amino acid sequence of SEQ ID NO:2 or SEQ ID NO:3.

5. The composition of claim 1, wherein the polypeptide comprises an amino acid sequence that is at least 90% identical to the amino acid sequence of SEQ ID NO:2 or SEQ ID NO:3.

6. (canceled)

7. The composition of claim 1 wherein the one or more other cellulases are selected from no or one or more other beta-glucosidases, one or more cellobiohydrolases, and one or more endoglucanases.

8. (canceled)

9. The composition of claim 1, wherein the one or more hemicellulases are selected from one or more xylanases, one or more beta-xylosidases, and one or more α-L-arabinofuranosidases.

10-13. (canceled)

14. A host cell comprising an expression vector comprising a recombinant nucleic acid encoding a recombinant polypeptide comprising an amino acid sequence that is at least 80% identical to the amino acid sequence of SEQ ID NO:2 or SEQ ID NO:3, wherein the polypeptide has beta-glucosidase activity.

15. The host cell of claim 14, wherein the host cell is a bacterial cell or a fungal cell.

16. A composition comprising the host cell of claim 14 and a culture medium.

17. A method of producing a beta-glucosidase, comprising: culturing the host cell of claim 14 in a culture medium, under suitable conditions to produce the beta-glucosidase.

18. (canceled)

19. A method for hydrolyzing a lignocellulosic biomass substrate, comprising: contacting the lignocellulosic biomass substrate with the composition of claim 15, to yield a glucose and other sugars.


SEQUENCE LISTING (51 sequences)

SEQ ID NO: 1 - DNA, 2871 bp, Melanocarpus albomyces
  misc_feature: Genomic DNA sequence encoding Mal3A
atgaaggccgctcttgcggttgcctcgctgctcagcggcagtcttgctgccgcgggcacgctccatccacgacacaaggtacggcaagctcgccgttccctggctggtgatggttcgggagtgagagtcccgtttgctgacagtcaataaaatcacccatcagctcgcaaagagggccctcgcaacgtcggatcccttttacccctcgccatggatgaatcccgaggcagatggctgggcggaagcgtacgcccaggcgagggagttcgtctcgcagatgacgctgctggagaaggtcaacctgaccaccggcaccgggtaagtgtcgcgggtggccgcccatacccgtggcgcctttcttctccatgcacgatgcgccgtgattctgacgattcgcagctgggcgtccgagcagtgcgtgggcaacacaggctcaattcctcgcctcggtctccgcagcttgtgcttgcacgacgctccgcttggcatccgcgggtcggactacaactcggccttcccctcgggacagaccgtcgccgccacctttgaccgcactctgatgtacaggcgcggctacgccatgggcctcgaggcgaagggcaagggcatcaacgtcctgctcgggccgtccgctggccccatcggccgcatgcctgccggcggccggaactgggaaggcttctcgccggatcccgtgctctcgggcattggcatggccgagtcggtcaagggcatccaggacgccggcgtgatcgcctgcgccaagcacttcatcggcaacgagcaaggtaagtcgggtcgacacggccgcgggatatggggtggtggtggtggtggtggtgatggagagcccgccagctgacatggggatcagagcacttcagacaggtgggagaggccatcgggtacggcttcgacatcaccgagacgctgtcttccaacatcgacgacaggacgatgcacgagctctacctctggcccttcgcggatgctgtccgcgcgggcgtcggctccatcatgtgctcgtaccagcaggtcaacaactcgtatgcctgccagaactccaaggtcctgaacgacctgctcaagaacgagctcggattccagggcttcgtcctgagcgactggcaagcgcagcacacgggcgcggccagcgccgtcgccggtctcgacatgaccatgccgggagacaccgagttcaacacgggcctcagctactggggcaccaacctcacgctcgccgtgctgaacggcaccgtcccggcctaccggatcgatgacatggccatgcgcatcatggccgccttcttcaaggtcagcaagagcatcgacctggaccccatcaacttctccttctggacgctggacacgtacggcccgatccactgggccgcgaacgagggccaccagcagatcaaccaccacgtcgacgtccggcagcccgaccacgcacacctcatccgcgagatcggcgccaagggcacggtgctgctgaagaacacggggtctctgcccctcgacaagcccaagttcctggccgtcatcggcgaggacgccggcccgaaccccaggggccccaactcctgcgccgaccgcggctgcaacgagggcacgctcgccatgggctggggctcgggcacggccaacttcccgtacctggtgacgccggatgcggcgctgcaggcccaggccatccaggacggctcgcgatacgagagcatcctgtccaactacgcgctcgatgagacgagggccctggtgtcgcaggaggatgccaccgcgatcgtcttcgtcaatgccaactcgggcgagggctacatcaacgtggacggcaacatgggcgaccgcaagaacctgacgctctggcggggcggcgacgacctggtcaagaacgtgtcgagctggtgctccaacaccatcgtcgtcatccactccaccggccccgtcctcctgacagactggtacgacagccccaacatcacggcgatcctgtgggccggcctccccggccaggagtcgggcaactcgatcgtcgatgtcctgtacggcaaggtcaacccggccggccgcacgcccttcacgtggggagcgacccgggagggctacggcgccgacgttctctacgagccgaacaacggcaacggcgcgccgcagcaggacttcaccgagggcgtcttcatcgactaccgctacttcgacaaggccaacacgtcggtcatctacgagttcggccacggcctcagctacacgacgttcgagtacagcaacatccgggtggagaagcacaacgccggcccgtaccggccgacggagggcatgacggcgcccgcgccgacgtttggcaacttctcgaccgacctcgaggactacctgttcccggaggacgagttcccctacgtctaccagtacatctacccgtacctcaacacgacggaccccgagaaggcgtcggccgatccgcactacggccaggcggccgacgagttcctgccgccgcgcgccaccgacgactcggcgcagccgctcctgcgcgcgtcggacaggcacgcgcccggcggcaaccgcggcctgtacgacgtgctgtacaccgtcacggccgacatcaccaacacgggccccatcgtcggggaggaggtgccgcagctctacgtctcgctcggcgggcccgacaaccccaaggtcgtcctccgcgactttgcccgcctgcgcatcgaccccggccagacggtccggttccgcggcaccctcacgaggagggacctgagcaactgggaccccgtcgtccaggactgggtcgtcggccgccacaacaagaccgtctatgtcggccggagcagccgcaagctggatctgagtgctgccttgccttga

SEQ ID NO: 2 - protein, 872 aa, Melanocarpus albomyces
  misc_feature: Polypeptide sequence of Mal3A
MKAALAVASLLSGSLAAAGTLHPRHKLAKRALATSDPFYPSPWMNPEADGWAEAYAQAREFVSQMTLLEKVNLTTGTGWASEQCVGNTGSIPRLGLRSLCLHDAPLGIRGSDYNSAFPSGQTVAATFDRTLMYRRGYAMGLEAKGKGINVLLGPSAGPIGRMPAGGRNWEGFSPDPVLSGIGMAESVKGIQDAGVIACAKHFIGNEQEHFRQVGEAIGYGFDITETLSSNIDDRTMHELYLWPFADAVRAGVGSIMCSYQQVNNSYACQNSKVLNDLLKNELGFQGFVLSDWQAQHTGAASAVAGLDMTMPGDTEFNTGLSYWGTNLTLAVLNGTVPAYRIDDMAMRIMAAFFKVSKSIDLDPINFSFWTLDTYGPIHWAANEGHQQINHHVDVRQPDHAHLIREIGAKGTVLLKNTGSLPLDKPKFLAVIGEDAGPNPRGPNSCADRGCNEGTLAMGWGSGTANFPYLVTPDAALQAQAIQDGSRYESILSNYALDETRALVSQEDATAIVFVNANSGEGYINVDGNMGDRKNLTLWRGGDDLVKNVSSWCSNTIVVIHSTGPVLLTDWYDSPNITAILWAGLPGQESGNSIVDVLYGKVNPAGRTPFTWGATREGYGADVLYEPNNGNGAPQQDFTEGVFIDYRYFDKANTSVIYEFGHGLSYTTFEYSNIRVEKHNAGPYRPTEGMTAPAPTFGNFSTDLEDYLFPEDEFPYVYQYIYPYLNTTDPEKASADPHYGQAADEFLPPRATDDSAQPLLRASDRHAPGGNRGLYDVLYTVTADITNTGPIVGEEVPQLYVSLGGPDNPKVVLRDFARLRIDPGQTVRFRGTLTRRDLSNWDPVVQDWVVGRHNKTVYVGRSSRKLDLSAALP

SEQ ID NO: 3 - protein, 856 aa, Melanocarpus albomyces
  misc_feature: Mature Mal3A polypeptide sequence
AAGTLHPRHKLAKRALATSDPFYPSPWMNPEADGWAEAYAQAREFVSQMTLLEKVNLTTGTGWASEQCVGNTGSIPRLGLRSLCLHDAPLGIRGSDYNSAFPSGQTVAATFDRTLMYRRGYAMGLEAKGKGINVLLGPSAGPIGRMPAGGRNWEGFSPDPVLSGIGMAESVKGIQDAGVIACAKHFIGNEQEHFRQVGEAIGYGFDITETLSSNIDDRTMHELYLWPFADAVRAGVGSIMCSYQQVNNSYACQNSKVLNDLLKNELGFQGFVLSDWQAQHTGAASAVAGLDMTMPGDTEFNTGLSYWGTNLTLAVLNGTVPAYRIDDMAMRIMAAFFKVSKSIDLDPINFSFWTLDTYGPIHWAANEGHQQINHHVDVRQPDHAHLIREIGAKGTVLLKNTGSLPLDKPKFLAVIGEDAGPNPRGPNSCADRGCNEGTLAMGWGSGTANFPYLVTPDAALQAQAIQDGSRYESILSNYALDETRALVSQEDATAIVFVNANSGEGYINVDGNMGDRKNLTLWRGGDDLVKNVSSWCSNTIVVIHSTGPVLLTDWYDSPNITAILWAGLPGQESGNSIVDVLYGKVNPAGRTPFTWGATREGYGADVLYEPNNGNGAPQQDFTEGVFIDYRYFDKANTSVIYEFGHGLSYTTFEYSNIRVEKHNAGPYRPTEGMTAPAPTFGNFSTDLEDYLFPEDEFPYVYQYIYPYLNTTDPEKASADPHYGQAADEFLPPRATDDSAQPLLRASDRHAPGGNRGLYDVLYTVTADITNTGPIVGEEVPQLYVSLGGPDNPKVVLRDFARLRIDPGQTVRFRGTLTRRDLSNWDPVVQDWVVGRHNKTVYVGRSSRKLDLSAALP

SEQ ID NO: 4 - protein, 744 aa, Trichoderma reesei
  misc_feature: T.reesei Bgl1 polypeptide sequence
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 5 - protein, 725 aa, Trichoderma reesei
  misc_feature: Mature T.reesei Bgl1 polypeptide sequence
DSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 6 - protein, 16 aa, Melanocarpus albomyces
  misc_feature: Mal3A signal peptide sequence
MKAALAVASLLSGSLA

SEQ ID NO: 7 - protein, 19 aa, Trichoderma reesei
  misc_feature: T. reesei Bgl1 signal peptide sequence
MRYRTAAALALATGPFARA

SEQ ID NO: 8 - protein, 32 aa, Artificial Sequence
  Synthetic signal peptide
MVSFTSLLAASPPSRASCRPAAEVESVAVEKR

SEQ ID NO: 9 - protein, 16 aa, Artificial Sequence
  Synthetic signal peptide
MKANVILCLLAPLVAA

SEQ ID NO: 10 - protein, 18 aa, Artificial Sequence
  Synthetic signal peptide
MIVGILTTLATLATLAAS

SEQ ID NO: 11 - protein, 17 aa, Artificial Sequence
  Synthetic signal peptide
MYRKLAVISAFLATARA

SEQ ID NO: 12 - protein, 23 aa, Artificial Sequence
  Synthetic signal peptide
MLLNLQVAASALSLSLLGGLAEA

SEQ ID NO: 13 - protein, 19 aa, Artificial Sequence
  Synthetic signal peptide
MKLNWVAAALSIGAAGTDS

SEQ ID NO: 14 - protein, 19 aa, Artificial Sequence
  Synthetic signal peptide
MASIRSVLVSGLLAAGVNA

SEQ ID NO: 15 - protein, 22 aa, Artificial Sequence
  Synthetic signal peptide
MWLTSPLLFASTLLGLTGVALA

SEQ ID NO: 16 - protein, 16 aa, Artificial Sequence
  Synthetic signal peptide
MRFSWLLCPLLAMGSA

SEQ ID NO: 17 - protein, 22 aa, Artificial Sequence
  Synthetic signal peptide
MRLLSFPSHLLVAFLTLKEASS

SEQ ID NO: 18 - protein, 20 aa, Artificial Sequence
  Synthetic signal peptide
MQLKFLSSALLLSLTGNCAA

SEQ ID NO: 19 - protein, 18 aa, Artificial Sequence
  Synthetic signal peptide
MKVYWLVAWATSLTPALA

SEQ ID NO: 20 - protein, 19 aa, Artificial Sequence
  Synthetic signal peptide
MVRFSSILAAAACFVAVES

SEQ ID NO: 21 - protein, 20 aa, Artificial Sequence
  Synthetic signal peptide
MIHLKPALAALLALSTQCVA

SEQ ID NO: 22 - protein, 17 aa, Artificial Sequence
  Synthetic signal peptide
MALQTFFLLAAAMLANA

SEQ ID NO: 23 - protein, 19 aa, Artificial Sequence
  Synthetic signal peptide
MKLNKPFLAIYLAFNLAEA

SEQ ID NO: 24 - protein, 20 aa, Artificial Sequence
  Synthetic signal peptide
MAPLSLRALSLLALTGAAAA

SEQ ID NO: 25 - protein, 19 aa, Artificial Sequence
  Synthetic signal peptide
MVRPTILLTSLLLAPFAAA

SEQ ID NO: 26 - protein, 21 aa, Artificial Sequence
  Synthetic signal peptide
MHMHSLVAALAAGTLPLLASA

SEQ ID NO: 27 - protein, 19 aa, Artificial Sequence
  Synthetic signal peptide
MVHLSSLAAALAALPLVYG

SEQ ID NO: 28 - protein, 17 aa, Artificial Sequence
  Synthetic signal peptide
MRFSLAATTLLAGLATA

SEQ ID NO: 29 - protein, 19 aa, Artificial Sequence
  Synthetic signal peptide
MVVLSKLVSSILFASLVSA

SEQ ID NO: 30 - protein, 19 aa, Artificial Sequence
  Synthetic signal peptide
MVQIKAAALAMLFASHVLS

SEQ ID NO: 31 - protein, 17 aa, Artificial Sequence
  Synthetic signal peptide
MKASSVLLGLAPLAALA

SEQ ID NO: 32 - protein, 19 aa, Artificial Sequence
  Synthetic signal peptide
MRFPSIFTAVLFAASSALA

SEQ ID NO: 33 - protein, 85 aa, Artificial Sequence
  Synthetic signal peptide
MRFPSIFTAVLFAASSALAAPVNTTTEDETAQIPAEAVIGYLDLEGDFDVAVLPFSNSTNNGLLFINTTIASIAAKEEGVSLDKR

SEQ ID NO: 34 - protein, 20 aa, Artificial Sequence
  Synthetic signal peptide
MLLQAFLFLLAGFAAKISAR

SEQ ID NO: 35 - DNA, 87 bp, Artificial Sequence
  Synthetic Sequence
atgataaaagtcccgcggttcatctgtatgatcgcgcttacatccagcgttctggcaagcggcctttctcaaagcgtttcagctcat

SEQ ID NO: 36 - protein, 29 aa, Artificial Sequence
  Synthetic signal peptide
MIKVPRFICMIALTSSVLASGLSQSVSAH

SEQ ID NO: 37 - DNA, 90 bp, Artificial Sequence
  Synthetic Sequence
atgaaaagaaagcttggtcgtcgccagttattaactggctttgttgcccttggcggtatggcgattacagctggtaaggcgcaggcttct

SEQ ID NO: 38 - protein, 30 aa, Artificial Sequence
  Synthetic signal peptide
MKRKLGRRQLLTGFVALGGMAITAGKAQAS

SEQ ID NO: 39 - DNA, 28 bp, Artificial Sequence
  Synthetic primer
caccatgagatatagaacagctgccgct

SEQ ID NO: 40 - DNA, 40 bp, Artificial Sequence
  Synthetic primer
cgaccgccctgcggagtcttgcccagtggtcccgcgacag

SEQ ID NO: 41 - DNA, 40 bp, Artificial Sequence
  Synthetic primer
ctgtcgcgggaccactgggcaagactccgcagggcggtcg

SEQ ID NO: 42 - DNA, 20 bp, Artificial Sequence
  Synthetic primer
cctacgctaccgacagagtg

SEQ ID NO: 43 - DNA, 20 bp, Artificial Sequence
  Synthetic primer
gtctagactggaaacgcaac

SEQ ID NO: 44 - DNA, 21 bp, Artificial Sequence
  Synthetic primer
gagttgtgaagtcggtaatcc

SEQ ID NO: 45 - DNA, 18 bp, Artificial Sequence
  Synthetic primer
caccatgaaggccgctct

SEQ ID NO: 46 - DNA, 20 bp, Artificial Sequence
  Synthetic primer
ctaaggcaaggcagcactca

SEQ ID NO: 47 - DNA, 20 bp, Artificial Sequence
  Synthetic primer
tgagagtcccgtttgctgac

SEQ ID NO: 48 - DNA, 19 bp, Artificial Sequence
  Synthetic primer
tcggtcaagggcatccagg

SEQ ID NO: 49 - DNA, 20 bp, Artificial Sequence
  Synthetic primer
ttcaaggtcagcaagagcat

SEQ ID NO: 50 - DNA, 20 bp, Artificial Sequence
  Synthetic primer
ctgacagactggtacgacag

SEQ ID NO: 51 - DNA, 20 bp, Artificial Sequence
  Synthetic primer
taccagtacatctacccgta